SEQUENCE LISTING (1 sequence)

SEQ ID NO: 1 - DNA, 66 bp, Mus musculus
  misc_feature 1..66: note = Description of sequence: DNA encoding mgll shRNA
  source 1..66: mol_type = genomic DNA
gctgatatgagccagctcatcataagttttggccactgactgacttatgatgctggctcatatcag

(19) United States
(12) Patent Application Publication
(10) Pub. No.: US 2026/0098268 A1
(43) Pub. Date: Apr. 9, 2026

(54) COMPOSITIONS AND METHODS TO TREAT ALZHEIMER'S DISEASE AND OTHER BRAIN DISEASES

(71) Applicant: THE BOARD OF REGENTS OF THE UNIVERSITY OF TEXAS SYSTEM, Austin, TX (US)

(72) Inventors: Chu CHEN, Austin, TX (US); Jian ZHANG, Austin, TX (US)

(21) Appl. No.: 19/115,421

(22) PCT Filed: Sep. 29, 2023
(86) PCT No.: PCT/US2023/075616

§ 371 Date: Mar. 26, 2025

Related U.S. Application Data
(60) Provisional application No. 63/411,964, filed on Sep. 30, 2022.

(51) Int. Cl. C12N 15/113; A61K 48/00; C12N 15/86

(52) U.S. Cl. CPC C12N 15/1137; A61K 48/0058; C12N 15/86; C12N 2310/14; C12N 2320/32; C12N 2750/14143; C12N 2830/008

(57) ABSTRACT

Pharmacological or genetic inactivation of MAGL reduces neuroinflammation and neuropathology in animal models of AD. However, global inactivation of MAGL induces functional tolerance of CB IR, which reduces the effectiveness of pharmacotherapies. Evidence has shown that selective inactivation of MAGL in astrocytes, but not in neurons, reduces neuropathology and synaptic and cognitive impairments in TBI. In particular, results showed that inactivation of astrocytic MAGL attenuates AD neuropathology and prevents deterioration in LTP, spatial learning and memory in AD animals. This shows that neuroprotective effects of global MAGL inactivation largely result from limiting 2-AG degradation in astrocytes, rather than in neurons. Therefore, selective inactivation of astrocytic MAGL provides a better therapeutic outcome for AD, as this greatly minimizes the potential adverse effects resulting from global inactivation-induced disruption of 2-AG degradation in neurons and in other peripheral tissues. To this end, an AAV-mediated gene silencing approach to knock MAGL selectively in astrocytes is presented.

### RELATED APPLICATION

This PCT application claims priority to, and the benefit of, U.S. Provisional Patent Application No. 63/411,964, filed Sep. 30, 2022, entitled “COMPOSITIONS AND METHODS TO TREAT ALZHEIMER'S DISEASE AND OTHER BRAIN DISEASES,” which is incorporated by reference herein in its entirety.

### GOVERNMENT SUPPORT

This invention was made with Government Support under Grant Nos. R01NS076815 and RF1AG081362-01 awarded by the National Institutes of Health. The Government has certain right in the invention.

### REFERENCE TO SEQUENCE LISTING

The sequence listing submitted on Sep. 29, 2023, as an .XML file entitled “11024-012WO1_ST26.xml” created on Sep. 26, 2023, and having a file size of 2,353 bytes is hereby incorporated by reference pursuant to 37 C.F.R. § 1.52(e)(5).

### FIELD

The present disclosure relates vector compositions and method of use to treat and/or prevent neurological diseases and injuries.

### BACKGROUND

Alzheimer's disease (AD) is one of the most common causes of dementia in the elderly. However, there are no effective therapies currently available for prevention and treatment of AD. Although the etiology of AD is multifactorial and complex, accumulated evidence shows that neuroinflammation is a root cause of neurodegenerative diseases, including AD. Hence, resolving neuroinflammation is crucial for preventing development of AD or delaying progression of the disease.

### SUMMARY

The present disclosure relates to directing an engineered adeno-associated virus (AAV) vector to astrocytes to treat neurodegenerative diseases and methods for the manufacture and use thereof.

In one aspect, disclosed herein are engineered adeno-associated virus (AAV) vectors (such as, for example an AAV9 vector) or lentiviral (LV) vectors (including, but not limited to Human Immunodeficiency Virus-1 (HIV-1), HIV-2, Simian immunodeficiency virus (SIV), feline immunodeficiency virus (FIV)) comprising an astrocyte-specific promoter (such as, for example, a glial fibrillary acidic protein (gfap) promoter) and a nucleic acid encoding a monoacylglycerol lipase (mgll) shRNA.

Disclosed herein are the engineered AAV vectors or lentiviral vectors of any preceding aspect, wherein the nucleic acid comprises at least 70% sequence identity to SEQ ID NO: 1. Also disclosed herein are the engineered AAV vectors or lentiviral vectors of any preceding aspect, wherein the nucleic acid comprises at least 80% sequence identity to SEQ ID NO: 1. Also disclosed herein are the engineered AAV vectors or lentiviral vectors of any preceding aspect, wherein the nucleic acid comprises at least 90% sequence identity to SEQ ID NO: 1. Also disclosed herein are the engineered AAV vectors or lentiviral vectors of any preceding aspect, wherein the nucleic acid comprises SEQ ID NO: 1.

Also disclosed herein are the engineered AAV vectors or lentiviral vectors of any preceding aspect, wherein the mgll shRNA inhibits expression of an monoacylglycerol lipase (MAGL) protein. In another aspect, disclosed herein is the engineered AAV vectors or LV vectors of any preceding aspect, wherein the vector further comprises a reporter gene (such as, for example green fluorescent protein (GFP) gene).

Also disclosed herein is the engineered AAV vectors of any preceding aspect, wherein the vector crosses a blood-brain-barrier (BBB).

In one aspect, disclosed herein is a methods of treating, inhibiting, reducing, decreasing, ameliorating, and/or preventing neurological diseases (such as for example, a neurodegenerative disease including, but not limited to Alzheimer's disease, Parkinson's disease, Huntington's disease, Amyotrophic lateral sclerosis (ALS). Friedreich ataxia, Frontotemporal dementia, Lewy body disease, Spinal muscular atrophy, Multiple sclerosis (MS), Batten disease, Creutzfeldt-Jakob disease, prion diseases, and/or neuropsychiatric disorders, including seizure/epilepsy and mood disorders) or traumatic brain injury (e.g., chronic traumatic encephalopathy, CTE) in a subject in need thereof, wherein the method comprises administering to the subject the engineered AAV or LV vectors of any preceding aspect. For example, disclosed herein are methods of treating, inhibiting, reducing, decreasing, ameliorating, and/or preventing a neurological disease (such as for example, a neurodegenerative disease including, but not limited to Alzheimer's disease, Parkinson's disease, Huntington's disease, Amyotrophic lateral sclerosis (ALS), Friedreich ataxia, Frontotemporal dementia, Lewy body disease, Spinal muscular atrophy, Multiple sclerosis (MS), Batten disease, Creutzfeldt-Jakob disease, prion diseases, and/or neuropsychiatric disorders, including seizure/epilepsy and mood disorders) or a traumatic brain injury (e.g., chronic traumatic encephalopathy, CTE) in a subject in need thereof, wherein the method comprises administering to the subject an engineered adeno-associated virus (AAV) vector (such as, for example an AAV9 vector) comprising an astrocyte-specific promoter (such as, for example, a glial fibrillary acidic protein (gfap) promoter) and a nucleic acid encoding a monoacylglycerol lipase (mgll) shRNA.

Disclosed herein are methods of treating, inhibiting, reducing, decreasing, ameliorating, and/or preventing a neurological disease of any preceding aspect, wherein the nucleic acid comprises at least 70% sequence identity to SEQ ID NO: 1. Also disclosed herein are methods of treating, inhibiting, reducing, decreasing, ameliorating, and/or preventing a neurological disease of any preceding aspect, wherein the nucleic acid comprises at least 80% sequence identity to SEQ ID NO: 1. Also disclosed herein are methods of treating, inhibiting, reducing, decreasing, ameliorating, and/or preventing a neurological disease of any preceding aspect, wherein the nucleic acid comprises at least 90% sequence identity to SEQ ID NO: 1. Also disclosed herein are methods of treating, inhibiting, reducing, decreasing, ameliorating, and/or preventing a neurological disease of any preceding aspect, wherein the nucleic acid comprises SEQ ID NO: 1.

Also disclosed herein are methods of treating, inhibiting, reducing, decreasing, ameliorating, and/or preventing a neurological disease of any preceding aspect, wherein the mgll shRNA inhibits expression of a monoacylglycerol lipase (MAGL) protein. In some aspects, the vector further comprises a reporter gene (such as, for example, a green fluorescent protein (GFP) gene). In another aspect, disclosed herein is a method of treating, inhibiting, reducing, decreasing, ameliorating, and/or preventing neurological diseases of any preceding aspect, wherein the vector crosses a blood-brain-barrier (BBB) by systemic administration (i.e., through intravenous route).

Also disclosed herein are methods of treating, inhibiting, reducing, decreasing, ameliorating, and/or preventing neurological diseases of any preceding aspect, wherein a pharmaceutically effective amount of the vector is administered through a bodily fluid of the subject. In another aspect, disclosed herein is a method of any preceding aspect, wherein the vector is further administered by an intravenous, intracerebroventricular (ICV), or intrathecal injection or nasal application. The vector is also administered via nanoparticles or extracellular vesicles (EVs, i.e., exosome).

In one aspect, disclosed herein is a methods of improving, increasing, retaining, and/or recovering spatial learning and memory retention in a subject with a neurological disease (such as for example, a neurodegenerative disease including, but not limited to Alzheimer's disease, Parkinson's disease, Huntington's disease, Amyotrophic lateral sclerosis (ALS), Friedreich ataxia, Lewy body disease, Spinal muscular atrophy, Multiple sclerosis (MS), Batten disease, Creutzfeldt-Jakob disease, prion diseases, and/or neuropsychiatric disorders, including seizure/epilepsy and mood disorders) or traumatic brain injury in a subject in need thereof, wherein the method comprises administering to the subject the engineered AAV vectors of any preceding aspect. For example, disclosed herein are methods of improving, increasing, retaining, and/or recovering spatial learning and memory retention in a subject with a neurological disease (such as for example, a neurodegenerative disease including, but not limited to Alzheimer's disease, Parkinson's disease, Huntington's disease, Amyotrophic lateral sclerosis (ALS), Friedreich ataxia, Lewy body disease, Spinal muscular atrophy, Multiple sclerosis (MS), Batten disease, Creutzfeldt-Jakob disease, prion diseases, and/or neuropsychiatric disorders, including seizure/epilepsy and mood disorders) or a traumatic brain injury in a subject in need thereof, wherein the method comprises administering to the subject an engineered adeno-associated virus (AAV) vector (such as, for example an AAV9 vector) or lentiviral vectors (including, but not limited to Human Immunodeficiency Virus-1 (HIV-1), HIV-2, Simian immunodeficiency virus (SIV), feline immunodeficiency virus (FIV)) comprising an astrocyte-specific promoter (such as, for example, a glial fibrillary acidic protein (gfap) promoter) and a nucleic acid encoding a monoacylglycerol lipase (mgll) shRNA.

Also disclosed herein are improving, increasing, retaining, and/or recovering spatial learning and memory retention in a subject with a neurological disease of any preceding aspect, wherein the mgll shRNA inhibits expression of a monoacylglycerol lipase (MAGL) protein. In some aspects, the vector further comprises a reporter gene (such as, for example, a green fluorescent protein (GFP) gene).

Disclosed herein are methods of improving, increasing, retaining, and/or recovering spatial learning and memory retention in a subject of any preceding aspect, wherein the nucleic acid comprises at least 70% sequence identity to SEQ ID NO: 1. Also disclosed herein are methods of improving, increasing, retaining, and/or recovering spatial learning and memory retention in a subject of any preceding aspect, wherein the nucleic acid comprises at least 80% sequence identity to SEQ ID NO: 1. Also disclosed herein are methods of improving, increasing, retaining, and/or recovering spatial learning and memory retention in a subject of any preceding aspect, wherein the nucleic acid comprises at least 90% sequence identity to SEQ ID NO: 1. Also disclosed herein are methods of improving, increasing, retaining, and/or recovering spatial learning and memory retention in a subject of any preceding aspect, wherein the nucleic acid comprises SEQ ID NO: 1.

In some aspects, the vector crosses the blood-brain-barrier (BBB). In some aspect, the vector targets an astrocyte. In some aspects, the vector does not target a neuron. In some aspects, disclosed herein is a method of improving, increasing, retaining, and/or recovering spatial learning and memory retention in a subject with a neurological disease of any preceding aspect, wherein the vector crosses a blood-brain-barrier (BBB).

Also disclosed herein are methods of improving, increasing, retaining, and/or recovering spatial learning and memory retention in a subject with a neurological disease or a traumatic brain injury of any preceding aspect, wherein a pharmaceutically effective amount of the vector is administered through a bodily fluid of the subject. In another aspect, disclosed herein is a method of any preceding aspect, wherein the vector is further administered by an intravenous, intracerebroventricular (ICV), or intrathecal injection or nasal application.

### BRIEF DESCRIPTION OF THE DRAWINGS

The accompanying drawings, which are incorporated in and constitute a part of this specification, illustrate certain examples of the present disclosure and together with the description, serve to explain, without limitation, the principles of the disclosure. Like numbers represent the same elements throughout the figures.

FIG. 1 shows the diagram of 2-Arachidonoylglycerol (2-AG) synthesis and metabolism.

FIG. 2 shows that selective inactivation of MAGL in astrocytes does not induce CB1R desensitization. Whole-cell patch clamp recordings were made in hippocampal CA1 pyramidal neurons to detect GABAergic synaptic response to WIN (5 μM) in WT, tKO, nKO and aKO. RIM (2 μM) was used. *P<0.05, **P<0.01 compared with WT, § § P<0.01 compared with tKO or nKO (ANOVA with the Bonferroni post-hoc test, 6-7 recordings/3-4 mice).

FIGS. 3A, 3B, 3C, and 3D show that the inactivation of MAGL in neurons impairs learning and memory. FIG. 3A shows the NOR test that was conducted in 3-mo-old WT, tKO, nKO, and aKO mice. ***p<0.001 compared with WT, § § P<0.01 compared with tKO, ###P<0.001 compared with nKO (ANOVA with Bonferroni post hoc test, n=15 to 16 mice/group). FIG. 3B shows the protocol for injection of AAV-syn-cre vectors in 3-mo-old mice. FIG. 3C shows the NOR test in mgll^{flox/flox} mice that received AAV9-Syn-Cre vectors. The test was performed 30 days after AAV vector injection. ***p<0.001 (ANOVA with Bonferroni post hoc test, n=12 to 14 mice/group). FIG. 3D shows the MWM test in mgll^{flox/flox} mice that received AAV9-Syn-Cre vectors. **p<0.01(ANOVA with repeated measures, n=11 to 13 mice/group).

FIG. 4 shows that the expression of MAGL in astrocytes is elevated in the hippocampus of both patients with AD and animal models of AD. Immunoreactivity of MAGL and GFAP was assessed in the hippocampus of patients with AD and normal subjects, 6-mo-old 5×FAD TG, 7-mo-old PS19 TG mice and their WT controls. Human postmortem samples were provided by NIH/Harvard Brain Tissue Resource Center. ***P<0.001 compared with normal subjects or WT mice, unpaired Student's t-test (n=4 subjects or mice/group). Scale bars: 40 μm.

FIGS. 5A, 5B, 5C, 5D, and 5E show that the overexpression of MAGL in astrocytes aggravates AD neuropathology and accelerates cognitive decline in 4-month-old 5×FAD-TG mice. FIG. 5A shows the AAV5-GFAP-hmgll-eGFP or AAV5-GFAP-eGFP control vectors that were stereotaxically injected into the hippocampus of APP-TG mice at 3 months of age. FIG. 5B shows the expression of human MAGL in astrocytes. Scale bars: 20 m. FIG. 5C shows the ELISA analysis of IL-1β and Aβ42 in hippocampal tissues from APP-TG mice 30 day after AAV injection. (Unpaired Student's t-test, n=6 mice/group). FIG. 5D shows the NOR test that was performed in WT and APP-TG mice 30 days after injection of AAV vectors. ***P<0.001 (ANOVA with Bonferroni post hoc test, n=13-17 mice/group). FIG. 5E shows the MWM test that was conducted 30 days after injection of AAV. **P<0.01 (ANOVA with repeated measures). The probe test was performed 24 hrs. after 7-day learning acquisition (ANOVA with Bonferroni post hoc test, n=14 to 17 mice/group).

FIG. 6 shows that the inactivation of MAGL in astrocytes decreases reactivity of microglia in 5×FAD TG mice. Immunoreactivity of Ibal in the brain was assessed in six-month-old APP-TG, APP-TG-nKO, and APP-TG-aKO mice. ***P<0.001 compared with APP-TG mice, ANOVA with the Bonferroni post-hoc test (n=5 mice/group). Scale bars: 200 μm.

FIG. 7 shows that the inactivation of MAGL in astrocytes decreases Aβ plaques in 5×FAD APP-TG mice. Immunostaining of 4G8 was assessed in 6-month-old APP-TG, APP-TG-tKO, APP-TG-nKO, and APP-TG-aKO mice. *P<0.05, ***P<0.001 compared with APP-TG; § § P<0.01, § § § P<0.001 compared with APP-TG-tKO; ###P<0.001 compared with APP-TG-nKO (ANOVA with the Bonferroni post-hoc test, n=5 mice/group). Scale bars: 200 μm.

FIGS. 8A and 8B show that the inactivation of MAGL in astrocytes limits Aβ processing and reduces Aβ42 in APP-TG mice. FIG. 8A shows the expression of enzymes synthesizing and degrading Aβ in hippocampal tissues from 6-month-old APP-TG, TG-tKO, TG-nKO, and TG-aKO mice. Data were normalized to APP-TG. *P<0.05, **P<0.01, ***P<0.001 compared with APP-TG; § § P<0.01, § § § P<0.001 compared with TG-tKO; #P<0.05, ##P<0.01, ###P<0.001 compared with TG-aKO (ANOVA with the Fisher's PLSD post-hoc test, n=5 mice/group). FIG. 8B shows the ELISA analysis of hippocampal Aβ42 in 6-month-old APP-TG, TG-tKO, TG-nKO, and TG-aKO mice. Data were normalized to APP-TG. *P<0.05, ***P<0.001 compared with APP-TG; § § P<0.01 compared with TG-tKO; ###P<0.001 compared with TG-nKO (ANOVA with the Bonferroni post-hoc test, n=6 mice/group).

FIG. 9 shows that the genetic inactivation of MAGL in astrocytes, but not in neurons, prevents decline in long-term synaptic plasticity in 5×FAD TG mice. LTP at hippocampal CA3-CA1 synapses was recorded in 6-month-old WT, APP-TG, APP-TG-tKO, APP-TG-nKO, and APP-TG-aKO mice. The values in the bar graph are the average of potentiation at 56-60 min after HFS. **P<0.01, ***P<0.001 compared with WT; § § P<0.01, § § § P<0.001 compared with APP-TG; #P<0.05 compared with APP-TG-tKO; ††P<0.01 compared with APP-TG-nKO (ANOVA with the Bonferroni post-hoc test (n=12-14 slices/5-6 mice/group).

FIG. 10 shows that the inactivation of MAGL in astrocytes reduces tau phosphorylation in PS19 tau-TG mice. Expression of Tau-5, AT8, AC-tau, and p-GSK3P was assessed in the hippocampal tissues from 7-month-old Tau-TG and tau-TG-aKO mice. Data were normalized to tau-TG. **P<0.01, ***P<0.001 compared with Tau-TG (ANOVA with the Fisher's PLSD post-hoc test, n=5 mice/group).

FIGS. 11A and 11B show that the expression of MAGL in astrocytes is reduced in the mice injected with AAV5-mgll-shRNA vectors. Mice were stereotaxically injected with AAV5-GFAP-eGFP-mgll-shRNAmir (2) vectors or AAV5-GFAP-eGFP control vectors into the hippocampus.

FIG. 11A shows the IHC analysis of MAGL and GFAP was conducted 30 days after AAV vector injection (n=3/group). Scale bars: 20 μm. FIG. 11B shows the immunoblot analysis of MAGL expression in the hippocampus was performed in mice injected with the AAV vectors. **P<0.01 (n=4/group).

FIGS. 12A and 12B show that the decreases in expression of synaptic proteins in APP-TG mice are prevented by inactivation of MAGL in astrocytes. FIG. 12A shows that the 5×FAD mice at 4 months of age were stereotaxically injected with AAV5-GFAP-eGFP-mgll-shRNAmir (2) vectors or AAV5-GFAP-eGFP control vector. FIG. 12B shows that the immunoblot analysis was conducted two months after injection of AAV vectors. **P<0.01, ***P<0.001 compared with WT-AAV-Con; § § P<0.01 compared with TG-AAV-Con (ANOVA with the Fisher's PLSD post-hoc test, n=4/group).

FIGS. 13A, 13B, and 13C show that the knockdown of MAGL in astrocytes prevents developing cognitive deficits in tau-TG mice. FIG. 13A shows a diagram illustrating the protocol for AAV injection in PS19 tau-TG mice. FIG. 13B shows the NOR test that was conducted in 7-mo-old WT and tau-TG mice that received AAV5-GFAP-eGFP-mgll-shRNAmir (2) or control vectors 2 months prior to the test (ANOVA with Bonferroni post hoc test, n=12 to 15 mice/group). FIG. 13C shows the MWM test in WT and tau-TG mice that received AAV-mgll-shRNA or control vectors. The test was performed 2 months after injection of AAV vectors. **P<0.01, ***p<0.001(ANOVA with repeated measures). The probe test was performed 24 hrs. after 7-day learning acquisition (ANOVA with Bonferroni post hoc test, n=12 to 15 mice/group).

FIG. 14 shows experimental protocols.

FIGS. 15A, 15B, and 15C show that GFP is expressed in astrocytes, but not in neurons, by intracerebroventricular (ICV) injection of AAV/PHP.eB-GFAP-eGFP vectors (AAV-Con). FIG. 15A shows the schematic illustration of the experimental protocol. AAV-Con vectors were administered into the brain by stereotaxic ICV injection. Immunostaining was performed 30 days following injection of AAV vectors. FIG. 15B shows a representative brain section from an animal that from an animal that was intracerebroventricularly injected with AAV-Con vectors. AAV/PHP.eB-GFAP-eGFP vectors are well distributed in the brain from an animal that was stereotaxically injected with the vectors in the cerebral ventricles. GFP protein (green) represents AAV vector distribution in the brain. Scale bar: 400 μm. FIG. 15C shows that the AAV vectors are only transduced in astrocytes (GFAP, an astrocytic marker) as GFP (green) is well merged with GFAP (red), but not in neurons (NeuN, a neuronal marker in FIG. 15C. Scale bar: 40 μm.

FIGS. 16A, 16B, 16C, and 16D show that the knockdown of MAGL in astrocytes prevents cognitive decline in 5×FAD APP transgenic mice. Intracerebroventricular (ICV) injection of AAV-PHP.eB-GFAP-eGFP-mgll-shRNAmir(2) vectors silence mgll in astrocytes to prevent cognitive deterioration in 5×FAD APP TG mice, an animal model of Alzheimer's disease. FIG. 16A shows AAV-PHP.eB-GFAP-eGFP-mgll-shRNAmir(2) (AAV-mgll-shRNA) or AAV-PHP.eB-GFAP-eGFP (AAV-Con) vectors that were administered into the brain by ICV injection in wild type (WT) and 5×FAD APP-TG mice at four months of age. The sequence of mgll shRNAmir in the vector: 5′-GCTGATATGAGCCAGCTCATCATAAGTTTTGGCCACTGACTGACTGATGATGCTGCCTC ATATCAG-3′ (SEQ ID NO: 1). FIG. 16B shows the Morris water maze (MWM) test that was performed two months after injection of AAV vectors. FIG. 16C shows that the silencing of mgll in astrocytes significantly improves spatial learning and memory by the MWM test in 6-month-old APP-TG mice that received AAV-mgll-shRNA vectors. ***P<0.001 compared with WT-AAV-Con, § § § P<0.001 compared with APP-TG-AAV-Con (ANOVA with repeated measures, n=11 to 15 mice/group). FIG. 16D shows that the knockdown of mgll in astrocytes improves memory retention by the probe trial test in APP-TG mice that received AAV-mgll-shRNA vectors. (ANOVA with Bonferroni post hoc test, n=11 to 15 mice/group). *P<0.05, compared with WT-AAV-Con, § § P<0.01 compared with APP-TG-AAV-Con (ANOVA with Bonferroni post hoc test, n=11 to 15 mice/group).

### DETAILED DESCRIPTION

The following description of the disclosure is provided as an enabling teaching of the disclosure in its best, currently known embodiment. To this end, those skilled in the relevant art will recognize and appreciate that many changes can be made to the various embodiments of the invention described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

### Definitions

In this specification and in the claims which follow, reference will be made to a number of terms which shall be defined to have the following meanings:

As used herein, the singular forms “a,” “an” and “the” include plural referents unless the context clearly dictates otherwise. Thus, for example, reference to a “metal” includes examples having two or more such “metals” unless the context clearly indicates otherwise.

Ranges can be expressed herein as from “about” one particular value, and/or to “about” another particular value. When such a range is expressed, another example includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

The term “comprising”, and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed.

“Optional” or “optionally” means that the subsequently described event or circumstance may or may not occur, and that the description includes instances where said event or circumstance occurs and instances where it does not.

The terms “about” and “approximately” are defined as being “close to” as understood by one of ordinary skill in the art. In one non-limiting embodiment the terms are defined to be within 10%. In another non-limiting embodiment, the terms are defined to be within 5%. In still another non-limiting embodiment, the terms are defined to be within 1%.

The term “administering” refers to an administration that is oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation or via an implanted reservoir. The term “parenteral” includes subcutaneous, intravenous, intramuscular, intra-articular, intra-synovial, intrasternal, intrathecal, intrahepatic, intralesional, and intracranial injections or infusion techniques.

As used herein, a “pharmaceutically effective amount” of a drug, composition, or vector refers to the necessary amount to achieve a therapeutic effect may vary according to factors such as the age, sex, and weight of the subject. Dosage regimens can be adjusted to provide the optimum therapeutic response. For example, several divided doses may be administered daily, or the dose may be proportionally reduced as indicated by the exigencies of the therapeutic situation.

An “increase” can refer to any change that results in a greater amount of a symptom, disease, composition, condition, or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

As used herein, the term, “deletion”, also called gene deletion, deficiency, or deletion mutation, refers to part of a chromosome or a sequence of DNA being left out during DNA replication. Deletion, or gene deletions can cause any number of nucleotides to be deleted from a single base to an entire piece of chromosome.

“Inhibitors” or “antagonist” of expression or of activity are used to refer to inhibitory molecules, respectively, identified using in vitro and in vivo assays for expression or activity of a described target protein, e.g., ligands, antagonists, and their homologs and mimetics. Inhibitors are agents that, e.g., inhibit expression or bind to, partially or totally block stimulation or activity, decrease, prevent, delay activation, inactivate, desensitize, or down regulate the activity of the described target protein, e.g., antagonists. As presented herein, the short hairpin RNA is an antagonist of the monoacylglycerol lipase (mgll) gene to prevent expression of the associated protein, MAGL. Control samples (untreated with inhibitors) are assigned a relative activity value of 100%. Inhibition of a described target protein is achieved when the activity value relative to the control is about 80%, optionally 50% or 25, 10%, 5%, or 1% or less.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

“Retain” and “retaining” mean to maintain or slow the decrease in an activity, response, or other biological parameter. This can include but is not limited to the complete maintenance of the activity, response, or parameter. This may also include, for example, a 10% reduction in any decrease in the activity, response, condition, or disease as compared to the native or control level. Thus, the reduction can be a 10, 20, 30, 40, 50, 60, 70, 80, 90, 100%, or any amount of reduction less than untreated levels.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

By “reduce” or other forms of the word, such as “reducing” or “reduction,” means lowering of an event or characteristic (e.g., tumor growth or disease progression). It is understood that this is typically in relation to some standard or expected value, in other words it is relative, but that it is not always necessary for the standard or relative value to be referred to. For example, “reduces bacterial growth” means reducing the rate of growth of a bacterium relative to a standard or a control.

By “recover” means to restore to native levels. Restoration of native levels of an activity, response, or other biological parameter can include complete restoration of prior functionality of the activity, response, or other biological parameter. Recover can also include an a 1, 2, 3, 4, 5, 6, 7, 8, 9, 10, 15, 20, 25, 30, 35, 40, 45, 50, 55, 60, 65, 70, 75, 80, 85, 90, 95, or 100% restoration to the native state of the activity, response, or biological parameter.

A “control” is an alternative subject or sample used in an experiment for comparison purpose. A control can be “positive” or “negative.” For example, where the purpose of the experiment is to determine a correlation of an altered expression level of a neurological gene or protein with a particular type of gene or protein altered from neurodegenerative, neuropsychiatric, and/or traumatic brain injury, it is generally preferable to use a positive control (a subject or a sample from a subject, carrying such alteration and exhibiting syndromes characteristic of that disease), and a negative control (a subject or a sample from a subject lacking the altered expression and clinical syndrome of that disease).

The terms “treat,” “treating,” “treatment,” and grammatical variations thereof as used herein, include partially or completely delaying, alleviating, mitigating, or reducing the intensity of one or more attendant symptoms of a disorder or condition and/or alleviating, mitigating, or impeding one or more causes of a disorder or condition. Treatments according to the disclosure may be applied preventively, prophylactically, palliatively, or remedially. Treatments are administered to a subject prior to onset (e.g., before obvious signs of neurodegenerative, neuropsychiatric, and/or traumatic brain injury), during early onset (e.g., upon initial signs and symptoms of neurodegenerative, neuropsychiatric, and/or traumatic brain injury), or after an established development of neurodegenerative, neuropsychiatric, and/or traumatic brain injury.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

A “promoter,” as used herein, refers to a sequence in DNA that mediates the initiation of transcription by an RNA polymerase. Transcriptional promoters may comprise one or more of a number of different sequence elements as follows: 1) sequence elements present at the site of transcription initiation; 2) sequence elements present upstream of the transcription initiation site and 3) sequence elements down-stream of the transcription initiation site. The individual sequence elements function as sites on the DNA, where RNA polymerases and transcription factors facilitate positioning of RNA polymerases on the DNA bind.

A “gene” refers to a polynucleotide containing at least one open reading irame mat is capaoie of encoding a particular polypeptide or protein after being transcribed and translated. Any of the polynucleotide sequences described herein may be used to identify larger fragments or full-length coding sequences of the gene with which they are associated.

The term “expression” as used herein refers to the process by which information from a gene is used in the synthesis of a functional gene product that enables it to produce a peptide/protein end product, and ultimately affect a phenotype, as the final effect.

As used herein a “viral vector” refers to a tool used in molecular biology to deliver genetic material (including DNA, RNA, and any other nucleic acid variations thereof) into a cell. This process is performed either inside a living organism or in cell culture. The viral genome is engineered to incorporate a desired gene or gene product, and following transduction, or transfer, of the virus into the host, said gene or gene product is expressed within the host.

A “shRNA” also termed a “short hairpin RNA” or “small hairpin RNA” is an artificial RNA molecule with a tight hairpin turn that is used to silence target gene expression. The turn within the artificial RNA molecule prevents or silences gene expression of the desired or target gene.

An “adeno-associated virus” or an “AAV” as used herein refers to a small virus belonging to the genus Dependoparvovirus which are replicative defective, non-enveloped viruses with linear single-stranded DNA. These viruses are commonly used for creating viral vectors for gene therapy, wherein said viruses can infect dividing and quiescent cells and persist in an extrachromosomal state without integrating into the host genome. AAVs can be engineered to express desired genes or gene products such as mRNA, shRNA, or miRNAs to overexpress or silence a target gene.

The term “serotype” refers to a distinct variation or variant within a viral genus or species usually classified based on expression of similar surface proteins. For example, the AAV genus, Dependoparvovirus, comprises serotypes including, but not limited to AAV1, AAV2, AAV3, AAV4, AAV5, AAV6, AAV7, AAV8, AAV9, AAV10, AAV11, and recombinant (rAAV).

The term “neurology” or “neurological” refers to the branch of medicine to treat disorders and diseases of the nervous system comprising nerve cells, or neurons and glial cells that form synapses, or signal transmission, in the nervous system.

A “neurodegenerative disease” is caused by the progressive loss of structure or function of neurons or glial cells, which make up the nervous system. These diseases include but are not limited to amyotrophic lateral sclerosis (ALS), multiple sclerosis, Parkinson's disease, Alzheimer's disease, Huntington's disease, Friedreich ataxia, Lewy body disease, Spinal muscular atrophy, Multiple sclerosis (MS), Frontotemporal dementia (FTD), Chronic traumatic encephalopathy (CTE). Batten disease, Creutzfeldt-Jakob disease, prion diseases, and neuropsychiatric disorders, including seizure/epilepsy and mood disorders. Neurodegenerative diseases can lead to cognitive and physical impairments, neuroinflammation (inflammation of the brain and spinal cord), and deterioration of brain and spinal cord tissues.

As used herein, a “traumatic brain injury” or “TBI” refers to an acquired brain injury that occurs when sudden physical injury damages the brain. TBI also results when the head of a subject suddenly and violently hits an object, or when an object pieces the skull and enter the brain tissue. Examples of TBI include, but are not limited to concussions, brain contusions, penetrating brain injuries, and anoxic brain injuries.

An “intracerebroventricular injection” or an “ICV” injection refers to an invasive injection technique wherein substances, compounds, compositions, vectors, and/or biological materials are delivered by a syringe needle directly into cerebrospinal fluid by way of the cerebral ventricles in order to bypass the blood-brain barrier.

The “blood-brain barrier” or the “BBB” refers to the highly selective semipermeable border of endothelial cells that prevents certain small molecules circulating in the blood from crossing into the extracellular fluid of the central nervous system.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### Compositions

Disclosed are the components to be used to prepare the compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular adeno-associated virus (AAV) or viral vector is disclosed and discussed and a number of modifications that can be made to a number of molecules including the AVV or viral vector discussed, specifically contemplated is each and every combination and permutation of AVV or viral vector and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

It is understood that the compositions disclosed herein have certain functions. Disclosed herein are certain structural requirements for performing the disclosed functions, and it is understood that there are a variety of structures which can perform the same function which are related to the disclosed structures, and that these structures will ultimately achieve the same result.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including matters of logic with respect to arrangement of steps or operational flow; plain meaning derived from grammatical organization or punctuation; and the number or type of embodiments described in the specification.

Alzheimer's disease (AD) is the most common cause of dementia in the elderly. However, no effective therapies are currently available for prevention and treatment of AD. Therefore, it is imperative to identify therapeutic targets and develop efficacious interventions for AD. While the etiology of AD is multifactorial and complex, accumulated evidence shows that neuroinflammation is a root cause of neurodegenerative diseases, including AD. Hence, resolving neuroinflammation is crucial for preventing development of AD or delaying progression of the disease.

Endocannabinoids are naturally occurring lipid mediators involved in a variety of physiological and pathological processes. 2-Arachidonoylglycerol (2-AG) is the most abundant endocannabinoid displaying profound anti-inflammatory and neuroprotective properties. 2-AG is primarily degraded by monoacylglycerol lipase (MAGL), the key enzyme that hydrolyzes 2-AG in the brain. However, global inactivation of MAGL may induce some adverse effects not only in peripheral organs, but also in the brain. For example, proper 2-AG metabolism in neurons is required for maintaining normal cognitive function. In addition, expression of MAGL in astrocytes is significantly elevated in the hippocampus of both patients with AD and animal models of AD and AAV-mediated overexpression of human MAGL in astrocytes aggravates neuropathology and accelerates cognitive decline in APP-TG mice, showing that 2-AG degradation in astrocytes promotes neuropathology in AD.

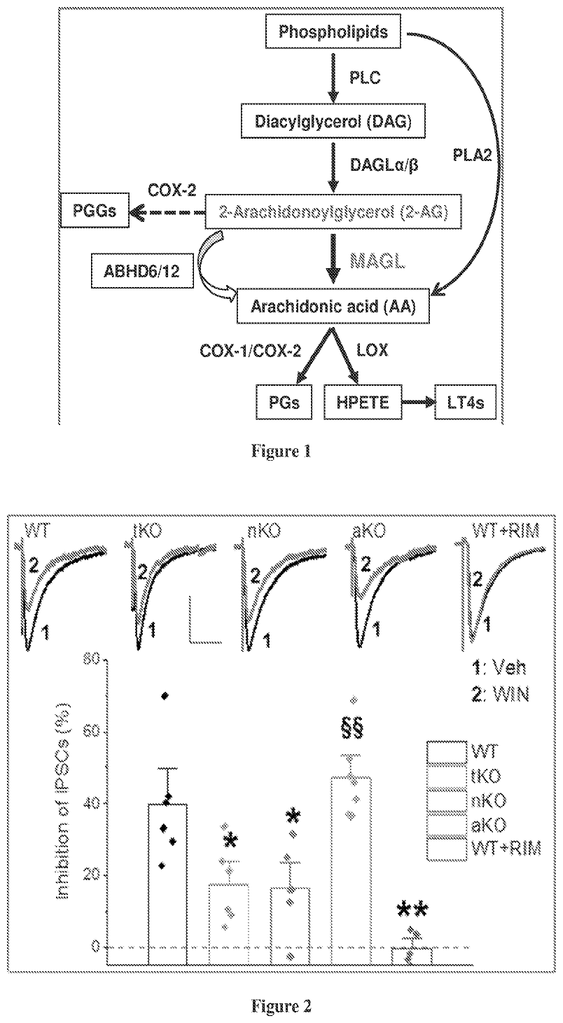

Endocannabinoid 2-AG synthesis and metabolism. Endocannabinoids are naturally occurring bioactive lipid mediators involved in a variety of physiological and pathological processes. Several lines of evidence indicate that endocannabinoids play an important role in maintaining brain homeostasis by modulating synaptic transmission and plasticity, resolving neuroinflammation, and protecting neurons from harmful insults. 2-Arachidonoylglycerol (2-AG) is the most abundant endogenous cannabinoid and full agonist for CB1 and CB2 receptors. It is largely synthesized from diacylglycerol (DAG) by diacylglycerol lipases (DAGLα and β) and hydrolyzed by monoacylglycerol lipase (MAGL), α/β hydrolase domain-containing protein 6 and 12 (ABHD6/12), and oxidatively metabolized by cyclooxygenase-2 (COX-2) when expression and activity of COX-2 are excessively elevated during inflammation (FIG. 1).

Although 2-AG is degraded by several enzymes, it has been estimated that 85% of 2-AG in the brain is hydrolyzed by MAGL, showing the important role of MAGL in degrading 2-AG in the brain. The immediate metabolite of 2-AG is arachidonic acid (AA), a precursor of prostaglandins converted by the enzymes COX-1/2 and of leukotrienes (LT4s: A4 to E4) catalyzed through the enzyme arachidonate 5-lipoxygenase (LOX, FIG. 1). Recent studies provided new insights into synthesis and metabolism of 2-AG in the brain. It has been revealed that 2-AG is synthesized in neurons and astrocytes primarily through DAGLα, while it is formed in microglial cells through DAGLβ. On the other hand, MAGL is the primary enzyme degrading 2-AG in neurons and astrocytes, whereas ABHD12 is the major enzyme degrading 2-AG in microglial cells. This is further supported by the fact that there are no differences in the 2-AG levels between WT and microglial MAGL knockout mice, showing that MAGL in microglia does not play a role in degrading 2-AG. These results show a diversity of 2-AG synthesis and metabolism in the brain.

Resolving neuroinflammation and protecting neurons by inhibition of 2-AU aegraaanon 2-AG displays anti-inflammatory and neuroprotective properties. Importantly, pharmacological inactivation or genetic deletion of MAGL alleviates neuroinflammation and neuropathology and improves synaptic and cognitive functions in several animal models of diseases, including epilepsy, Parkinson's disease, AD, and traumatic brain injury (TBI). In contrast, leukotrienes, and certain prostaglandins, which are metabolites of 2-AG, have long been known as proinflammatory and neurotoxic factors. Obviously, “a dual hit” occurs when MAGL is inactivated, resulting in augmentation of anti-inflammatory and neuroprotective 2-AG signaling, while lowering proinflammatory and neurotoxic eicosanoids. Thus, MAGL is a therapeutic target for neurodegenerative diseases, and many MAGL inhibitors have been developed for treatment of diseases. However, global inactivation of MAGL induces some adverse effects, which limits clinical applications of pharmacological MAGL inhibitors for AD.

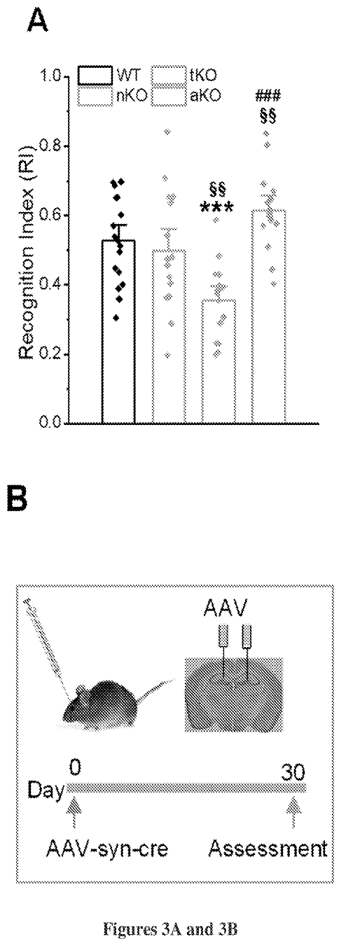

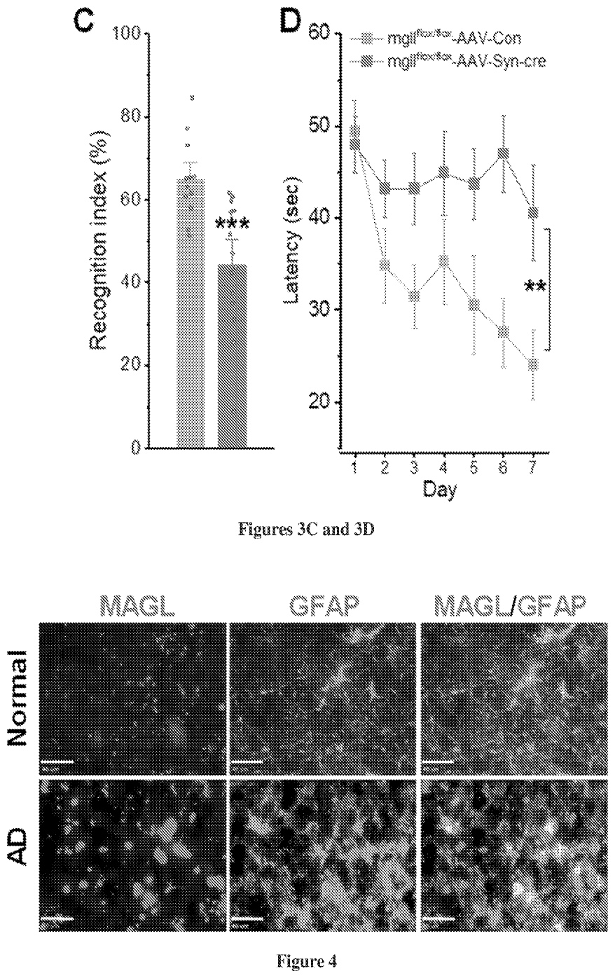

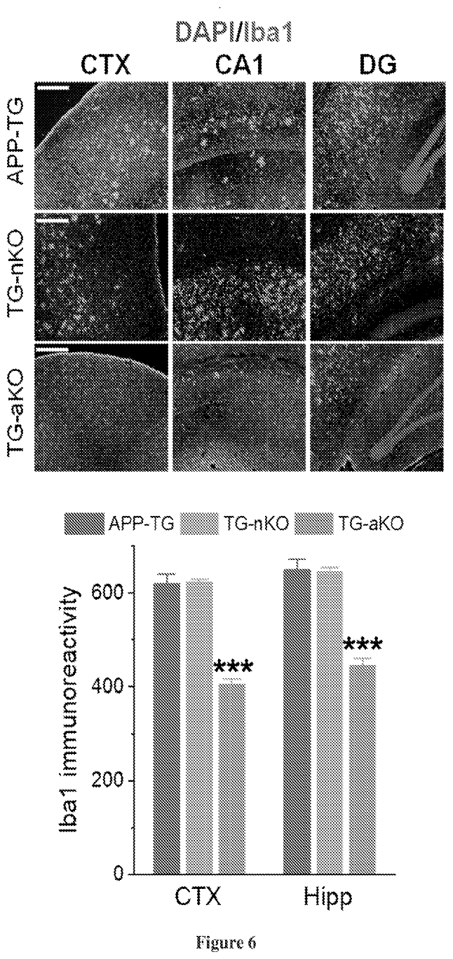

Adverse effects of global MAGL inactivation. Global inactivation of MAGL causes functional antagonism of the endocannabinoid system, primarily resulting from functional tolerance of CB1R, which reduces the effectiveness and efficacy of MAGL inhibitors. MAGL inactivation-induced functional tolerance of CB1R is largely due to inhibition of 2-AG metabolism in neurons, rather than in astrocytes (FIG. 2). Recent results showed that global inactivation of MAGL also induces other adverse effects, including increasing incidence of lung adenocarcinoma, worsening heart function after acute myocardial infarction, impairing fine motor coordination, and causing bone loss. Importantly, genetic inactivation of MAGL in neurons impairs spatial learning and memory retention. This cognitive impairment has been further confirmed (FIG. 3), showing that proper 2-AG degradation in neurons is important for maintaining normal cognitive function. In particular, genetic inactivation of MAGL in astrocytes, but not in neurons, significantly reduces neuropathology and improves synaptic functions in APP TG mice (FIGS. 6, 7, 8, 9, and 10), showing that neuroprotective effects produced by global MAGL inactivation in AD animals result primarily from limiting 2-AG degradation in astrocytes, rather than in neurons. Global inactivation MAGL using pharmacological inhibitors is not an optimal approach to achieve an ideal therapeutic goal for AD, as it will induce undesirable side effects resulting from disruption of 2-AG metabolism in neurons as well as in other peripheral tissues/cells.

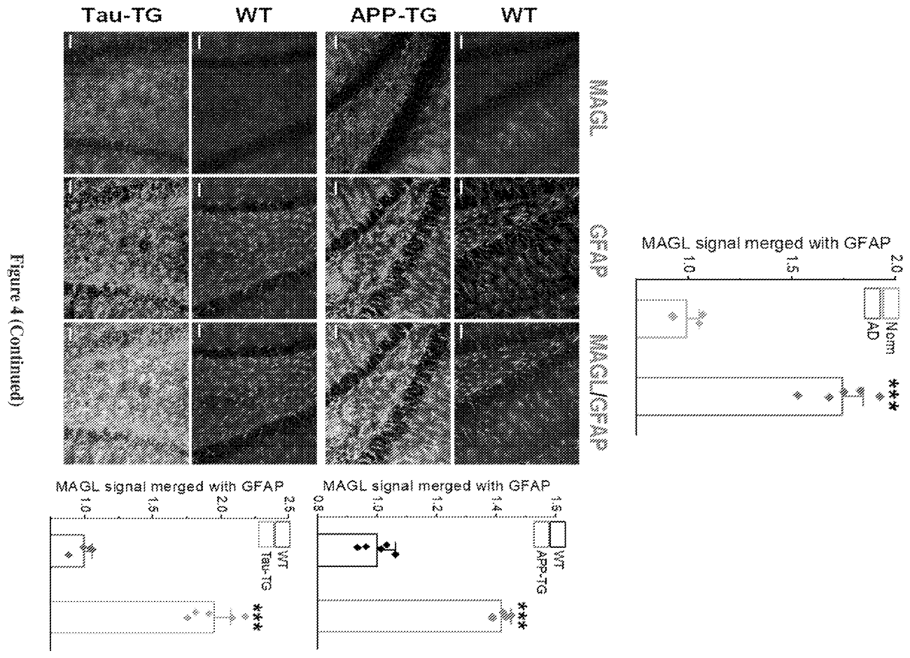

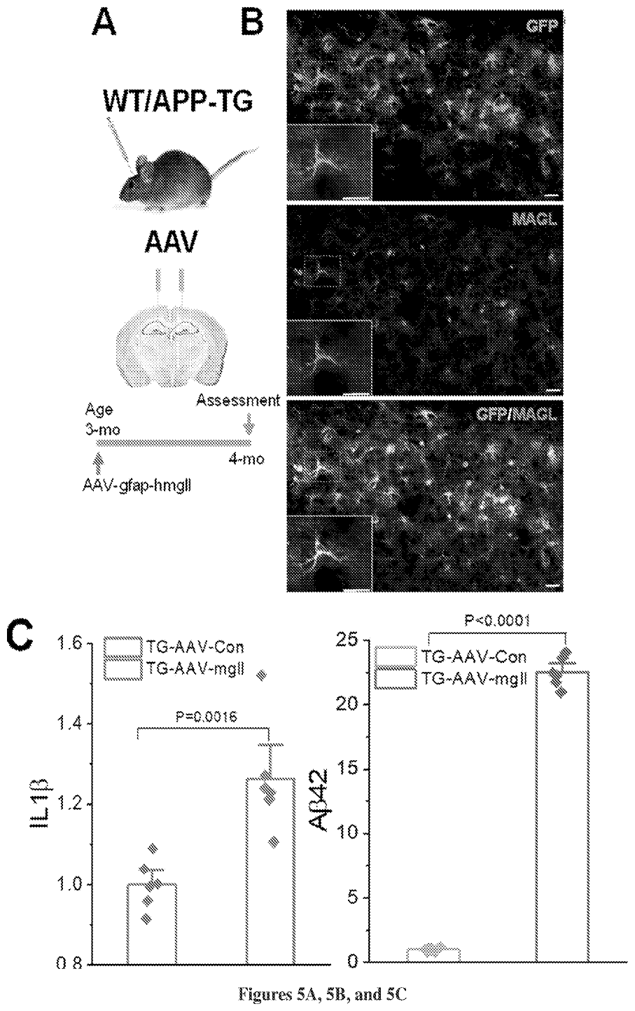

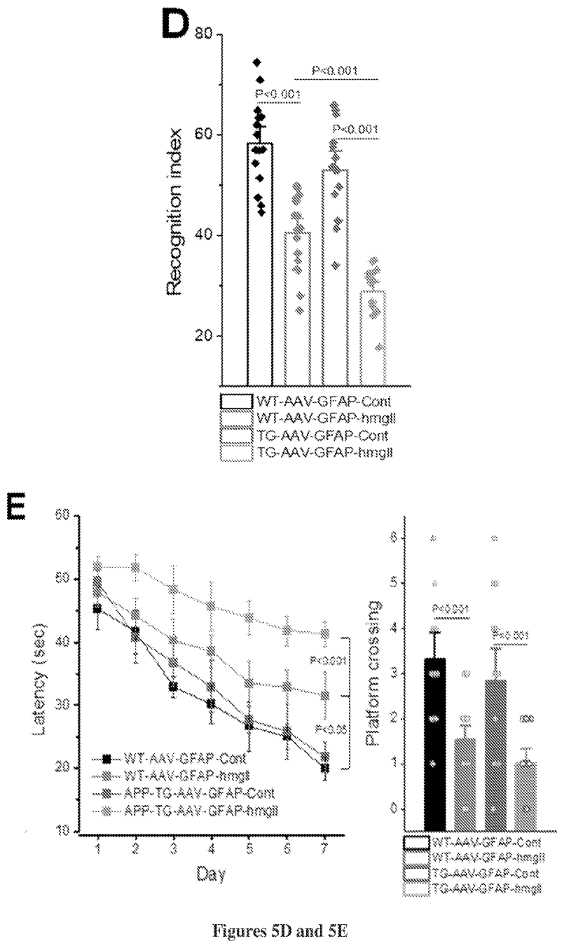

Astrocytic MAGL is a promising therapeutic target for AD. Activity or expression of MAGL is increased in patients with AD and in APP TG mice. Expression of MAGL in astrocytes is significantly elevated in the hippocampus of both patients with AD and animal models of AD (FIG. 4), showing that 2-AG degradation in astrocytes is escalated in AD, which promotes neuroinflammation and AD neuropathology. Indeed, AAV vector-mediated overexpression of human mgll in astrocytes facilitates IL-1β and Aβ formation and accelerates cognitive decline in APP-TG mice (FIG. 5), showing that elevated expression of MAGL in astrocytes will exacerbate AD neuropathology and accelerate cognitive deterioration in AD. Astrocytes have been recognized as an important player involved in a striking number of functions that are critically important for maintaining brain homeostasis in health and disease. Especially, astrocytes participate in neuroinflammatory and neurodegenerative processes that cause AD. Recent results provided clues of critical role of astrocytic 2-AG metabolism in resolving neuroinflammation. For instance, selective inactivation of MAGL in astrocytes mitigates LPS-induced inflammatory responses and prevents striatal neurodegeneration but does not cause CBIR desensitization. Single-cell transcriptomics analysis reveals that selective inactivation of MAGL in astrocytes, but not in neurons, enhances resilience to TBI-induced changes in expression of genes associated with inflammation or maintenance of brain homeostasis in astrocytes and microglia, which is important in alleviating TBI-induced neuropathology and deterioration in synaptic and cognitive functions. Proinflammatory prostaglandins in the brain are largely derived from 2-AG degraded by MAGL in astrocytes. Thus, inactivation of MAGL in astrocytes will not only augment anti-inflammatory 2-AG signaling, but also reduce the amount of 2-AG metabolites, including prostaglandins and leukotrienes, which are proinflammatory. Importantly, genetic inactivation of MAGL in astrocytes attenuates neuropathology in APP TG mice (FIGS. 6, 7, and 8) and improves long-term potentiation (LTP) (FIG. 9). In contrast, genetic deletion of MAGL in neurons fails to mitigate neuropathology and improve hippocampal LTP in APP-TG mice (FIGS. 6, 7, 8, and 9). Selective inactivation of MAGL in astrocytes will achieve a better therapeutic outcome for AD. However, current pharmacotherapies do not have the capacity to target a gene in a specific type of cells in the brain. The virus-mediated gene targeting technology provides such a platform for overexpression or silencing of a gene in a specific type of cells.

AAV vector-mediated silencing of astrocytic MAGL is an ideal and novel gene therapy for AD. Accumulated evidence shows that AAV vector-mediated gene replacement, silencing, and editing technologies provides a platform for the treatment of a variety of human diseases, including neurodegenerative diseases. Recent clinical results showed that AAV gene therapy is safe and effective. Therefore, use of the AAV vector-mediated silencing technology will allow to target MAGL specifically in astrocytes.

Astrocytic MAGL is an ideal therapeutic target for AD. MAGL is thought to be a therapeutic target for AD. However, recent results show that global inactivation of MAGL induces some adverse effects and reduce the efficacy of the MAGL inactivation by pharmacological inhibitors (e.g., antagonism of the endocannabinoid system). This shows that global inactivation of MAGL is not an optimal approach to achieve an ideal therapeutic goal for AD.

Use the AAV vector-mediated gene silencing strategy to knock MAGL down in astrocytes. Constraint of 2-AG degradation by knockdown of MAGL in astrocytes will not only achieve better therapeutic effects for AD, but also minimize potential adverse effects resulting from global MAGL inactivation-induced disruption of 2-AG metabolism in neurons and in other peripheral tissues. However, current pharmacotherapies lack the capacity to target a gene in a specific type of cells in the brain. Thus, use of AAV/PHP-eB vectors, an engineered AAV9-derived variant with the enhanced transduction efficiency, containing a gfap promoter to selectively silence MAGL in astrocytes as a novel and efficacious gene therapy to alleviate AD neuropathology and reverse deterioration in synaptic and cognitive functions in animal models of AD.

Adeno-associated virus (AAV) is a parvovirus having a simple single-stranded DNA genome that has no record of causing disease in humans. AAV is naturally defective requiring a helper virus, such as an adenovirus or herpes virus, to establish a productive infection. AAV infection is predictable with the AAV genome integrating on chromosome 19 contributing to increased interest to use AAV in gene therapy. Strategies in gene therapy emerged to generate AAV serotypes to drive cell specific, tissue specific, or temporal specific expression of genes of interest. Most recombinant AAV vectors are generated from the AAV2 vector template, wherein a mammalian promoter, a gene of interest, and a terminator sequence are placed between the inverted terminal repeat sequences (ITRs) of the AAV genome. The incorporated mammalian promoter determines how and when the gene of interest is expressed, therefore identifying the optimal promoter critical for the AAV gene therapy.

The disclosed vectors can also be derived from lentiviral vectors (including, but not limited to Human Immunodeficiency Virus-1 (HIV-1), HIV-2, Simian immunodeficiency virus (SIV), feline immunodeficiency virus (FIV)) which are well known in the art.

Strategies using single cell RNA sequencing analysis revealed that inactivation of MAGL in astrocytes suppresses TBI-induced expression of genes associated with inflammation in microglia and astrocytes. These results show that knockdown of MAGL in astrocytes provides a better therapeutic outcome for AD, as this approach greatly minimizes adverse effects resulting from global MAGL inactivation-induced disruption of 2-AG metabolism in neurons and in peripheral tissues. However, current pharmacotherapies do not have the capacity to target a molecule in a specific type of cells in the brain, as MAGL is expressed in many different types of cells. Collectively, these approaches illustrate the need to develop gene therapy strategies to regulate MAGL expression and 2-AG levels patients with neurodegenerative diseases.

Given the observation of elevated MAGL expression and unbalanced 2-AG metabolism in neurodegenerative disorders, such as Alzheimer's disease and traumatic brain injury events, there is need to address the aforementioned problems by developing strategies to regulate MAGL expression in astrocytes to treat neurodegenerative diseases.

The present disclosure relates to an engineered adeno-associated virus (AAV) vector that delivers a short hairpin RNA (shRNA) with an astrocyte-specific promoter to astrocytes for the treatment of neurodegenerative diseases and/or traumatic brain injuries and methods for the manufacture and use thereof.

A brain-specific promoter, such as for example an astrocyte-specific promoter, comprises a DNA sequence that binds specific proteins in the brain to initiate expression of a downstream gene. A downstream gene refers to the relative positions of genetic code of DNA or RNA, however since some genes on the same DNA strand molecule may be transcribed in opposite directions thus downstream areas of a molecule may change depending on which gene is used as the reference. A brain-specific promoter, such as for example an astrocyte-specific promoter, can also be cell-specific with expression being restricted to astrocytes (or glial cells). Accordingly, in one aspect, disclosed herein are engineered adeno-associated virus (AAV) vectors (such as, for example an AAV9 vector) comprising a brain specific promoter (such as for example, an astrocyte-specific promoter, including, but not limited to a glial fibrillaiy acidic protein (gfap) promoter) and a nucleic acid encoding a monoacylglycerol lipase (mgll) interfering RNA molecule (such as, for example, microRNA (miRNA), a small/short interfering RNA (siRNA), a short hairpin RNA (shRNA), or any variation of interfering RNA molecules).

As noted above, the disclosed vectors utilize an astrocyte specific promoter (such as, for example, the gfa promoter (gfap), including, but not limited to gfa2 or gfa28 promoters). It is understood and herein contemplated that other astrocyte promoters can be used in the disclosed AAV vectors including, but not limited to aldehyde dehydrogenase 1 family, member L1 (Aldh111) or solute carrier family 1 member 3 (slc1a3).

The engineered adeno-associated virus (AAV) vectors (such as, for example an AAV9 vector) comprises a nucleic acid encoding an RNA interfering molecule to disrupt translation of monoacylglycerol lipase (mgll) to a monoacylglycerol lipase (MAGL) protein. Many different approaches can be used to disrupt translation of a gene to a protein including, but not limited to the use of microRNA (miRNA), a small/short interfering RNA (siRNA), a short hairpin RNA (shRNA), or any variation of mgll interfering RNA molecules, shRNA molecules, double stranded interfering RNA molecules, are favored for gene therapy applications because they exhibit specific, long-lasting gene silencing of a target gene. In some embodiments, the engineered AAV vector encodes a double-stranded interfering RNA. In some embodiments, the engineered AAV vector encodes an microRNA (miRNA), a small/short interfering RNA (siRNA), a short hairpin RNA (shRNA), or any variation of interfering RNA molecules. Thus, disclosed herein are engineered adeno-associated virus (AAV) vectors (such as, for example an AAV9 vector) comprising an astrocyte-specific promoter (such as, for example, a glial fibrillary acidic protein (gfap) promoter) and a nucleic acid encoding a monoacylglycerol lipase (mgll) double-stranded interfering RNA wherein, the double-stranded interfering RNA is a mgll miRNA, a mgll siRNA, or a mgll shRNA. Accordingly, disclosed herein are engineered adeno-associated virus (AAV) vectors (such as, for example an AAV9 vector) comprising a brain specific promoters (such as for example, an astrocyte-specific promoter, including, but not limited to a glial fibrillary acidic protein (gfap) promoter) and a nucleic acid encoding a monoacylglycerol lipase (mgll) shRNA.

In some embodiments, the nucleic acid comprises a DNA molecule. In some embodiments, the nucleic acid comprises an RNA molecule.

In some embodiments, the nucleic acid comprises at least 50% sequence identity to SEQ ID NO: 1. In some embodiments, the nucleic acid comprises at least 55% sequence identity to SEQ ID NO: 1. In some embodiments, the nucleic acid comprises at least 60% sequence identity to SEQ ID NO: 1. In some embodiments, the nucleic acid comprises at least 65% sequence identity to SEQ ID NO: 1. In some embodiments, the nucleic acid comprises at least 70% sequence identity to SEQ ID NO: 1. In some embodiments, the nucleic acid comprises at least 75% sequence identity to SEQ ID NO: 1. In some embodiments, the nucleic acid comprises at least 80% sequence identity to SEQ ID NO: 1. In some embodiments, the nucleic acid comprises at least 85% sequence identity to SEQ ID NO: 1. In some embodiments, the nucleic acid comprises at least 90% sequence identity to SEQ ID NO: 1. In some embodiments, the nucleic acid comprises at least 95% sequence identity to SEQ ID NO: 1. In some embodiments, the nucleic acid comprises at least 99% sequence identity to SEQ ID NO: 1. In some embodiments, the nucleic acid comprises SEQ ID NO: 1.

In another aspect, disclosed herein is the engineered AAV vectors of any preceding aspect wherein the mgll shRNA inhibits expression of an monoacylglycerol lipase (MAGL) protein, AAV vectors can be used as for the basis of the disclosed engineered vectors as they offer many advantages over other viral vectors. For example, AAV type vectors can transport about 4 to 5 kb of genetic material. Vectors which contain this site-specific integration property are preferred. AAVs used can be derived from any AAV serotype, including but not limited to AAV1, AAV2, AAV3, AAV4, AAV5, AAV6, AAV7, AAV8, AAV9, AAV10, AAV11, and other types such as, for example AAV-Rh74, and/or synthetic AAV (such as, for example AAV-DJ, Anc80). AAV serotypes can be selected based on cell or tissue tropism. AAV vectors for use in the disclosed compositions and methods can be single stranded (SS) or self-complementary (SC).

Transcripts that are delivered via AAV vectors can be packaged as a linear single-stranded (ss) DNA with a length of approximately 4.7 kb (ssAAV) or as linear self-complementary (sc) DNA (scAAV). The benefit of the scAAV vector is that it contains a mutated inverted terminal repeat (ITR), which is required for replication and helps to bypass rate-limiting steps of second strand generation in comparison to ssDNA vectors. In another aspect, disclosed herein is the engineered AAV vector of any preceding aspect, wherein the vector is an AAV9 variant. The AAV vector can be further packaged into nanoparticles or extracellular vesicles, such as, for example, astrocyte-specific exosomes.

In another aspect, disclosed herein is the engineered AAV vector of any preceding aspect, wherein the vector further comprises a reporter gene. In some embodiments, the reporter gene is a luciferase, green fluorescent protein (GFP) gene, yellow fluorescent protein (YFP), blue fluorescent protein (BFP), cyane fluorescent protein (CFP), monomeric red fluorescent protein (mRFP), DiscosAoma striafa (DsRed), mCherry, mOrange, tdTomato, mSTrawberry, mPlum, photoactivatable GFP (PA-GFP), Venus, Kaede, monomeric kusabira orange (mKO), Dronpa, enhanced CFP (ECFP), Emerald, Cyan fluorescent protein for energy transfer (CyPet), super CFP (SCFP), Cerulean, photoswitchable CFP (PS-CFP2), photoactivatable RFP1 (PA-RFP1), photoactivatable mCherry (PA-mCherry), monomeric teal fluorescent protein (mTFP1), Eos fluorescent protein (EosFP), Dendra, TagBFP, TagRFP, enhanced YFP (EYFP), Topaz, Citrine, yellow fluorescent protein for energy transfer (YPet), super YFP (SYFP), enhanced GFP (EGFP), Superfolder GFP, T-Sapphire, Fucci, mKO2, mOrange2, mApple, Sirius, Azurite, EBFP, and/or EBFP2. In one aspect, disclosed herein are engineered AAV vectors comprising (such as, for example an AAV9 vector) comprising an astrocyte-specific promoter (such as, for example, a glial fibrillary acidic protein (gfap) promoter) and a nucleic acid encoding a monoacylglycerol lipase (mgll) shRNA further comprising a reporter gene of any preceding aspect (such as, for example, GFP).

In another aspect, disclosed herein is the engineered AAV vector of any preceding aspect, wherein the vector crosses a blood-brain-barrier (BBB).

In some embodiments, disclosed herein is the present disclosure of any preceding aspect, wherein the disclosure relates to directing a lentiviral vector to deliver a nucleic acid encoding a short hairpin RNA with a brain-specific promoter. In some embodiments, the lentiviral vector (LV) is a LV-gfap-mgll-shRNA vector.

### Methods

It is understood and herein contemplated that the disclosed engineered vectors provide a solution to the complications arising from global inactivation of MAGL through the use of small molecules or non-target specific gene therapy. In one aspect, disclosed herein is a methods of treating, inhibiting, reducing, decreasing, ameliorating, and/or preventing a neurological disease (such as for example, a neurodegenerative disease including, but not limited to Alzheimer's disease, Parkinson's disease, Huntington's disease, Amyotrophic lateral sclerosis (ALS), Friedreich ataxia, Lewy body disease, Spinal muscular atrophy, Multiple sclerosis (MS), Batten disease, Creutzfeldt-Jakob disease, prion diseases, and/or neuropsychiatric disorders, including seizure/epilepsy and mood disorders) or a traumatic brain injury in a subject in need thereof, wherein the method comprises administering to the subject any of the engineered viral vectors disclosed herein. For example, disclosed herein are methods of treating, inhibiting, reducing, decreasing, ameliorating, and/or preventing a neurological disease (such as for example, a neurodegenerative disease including, but not limited to Alzheimer's disease, Parkinson's disease, Huntington's disease, Amyotrophic lateral sclerosis (ALS), Friedreich ataxia, Lewy body disease, Spinal muscular atrophy, Multiple sclerosis (MS), Batten disease, Creutzfeldt-Jakob disease, prion diseases, and/or neuropsychiatric disorders, including seizure/epilepsy and mood disorders) or traumatic brain injury in a subject in need thereof, wherein the method comprises administering to the subject an engineered adeno-associated virus (AAV) vector (such as, for example an AAV9 vector) comprising a brain specific promoter (such as for example an astrocyte-specific promoter, including, but not limited to a glial fibrillary acidic protein (gfap) promoter) and a nucleic acid encoding a monoacylglycerol lipase (mgll) double-stranded interfering RNA (such as, for example, a microRNA (miRNA), a small/short interfering RNA (siRNA), a short hairpin RNA (shRNA), or any variation of interfering RNA molecules, including, but not limited to the double-stranded interfering RNA is a mgll miRNA, a mgll siRNA, or a mgll shRNA).

Another example, disclosed herein are methods of treating, inhibiting, reducing, decreasing, ameliorating, and/or preventing a neurological disease (such as for example, a neurodegenerative disease including, but not limited to Alzheimer's disease, Parkinson's disease, Huntington's disease, Amyotrophic lateral sclerosis (ALS). Friedreich ataxia, Lewy body disease. Spinal muscular atrophy, Multiple sclerosis (MS), Batten disease, Creutzfeldt-Jakob disease, prion diseases, and/or neuropsychiatric disorders, including seizure/epilepsy and mood disorders) or traumatic brain injury in a subject in need thereof, wherein the method comprises administering to the subject an engineered lentiviral (LV) vector (including, but not limited to Human Immunodeficiency Virus-1 (HIV-1), HIV-2, Simian immunodeficiency virus (SIV), feline immunodeficiency virus (FIV)) comprising a brain specific promoter (such as for example an astrocyte-specific promoter, including, but not limited to a glial fibrillary acidic protein (gfap) promoter) and a nucleic acid encoding a monoacylglycerol lipase (mgll) double-stranded interfering RNA (such as, for example, a microRNA (miRNA), a small/short interfering RNA (siRNA), a short hairpin RNA (shRNA), or any variation of interfering RNA molecules, including, but not limited to the double-stranded interfering RNA is a mgll miRNA, a mgll siRNA, or a mgll shRNA).

As noted above, the disclosed methods utilize an astrocyte specific promoter (such as, for example, the gfa promoter (gfap), including, but not limited to gfa2 or gfa28 promoters). It is understood and herein contemplated that other astrocyte promoters can be used in the disclosed AAV vectors including, but not limited to aldehyde dehydrogenase 1 family, member L1 (Aldh111) or solute carrier family 1 member 3 (slc1a3).

Disclosed herein are methods of treating, inhibiting, reducing, decreasing, ameliorating, and/or preventing a neurological disease, wherein the nucleic acid comprises at least 50% sequence identity to SEQ ID NO: 1. Also disclosed herein are methods of treating, inhibiting, reducing, decreasing, ameliorating, and/or preventing a neurological disease, wherein the nucleic acid comprises at least 55% sequence identity to SEQ ID NO: 1. Also disclosed herein are methods of treating, inhibiting, reducing, decreasing, ameliorating, and/or preventing a neurological disease, wherein the nucleic acid comprises at least 60% sequence identity to SEQ ID NO: 1. Also disclosed herein are methods of treating, inhibiting, reducing, decreasing, ameliorating, and/or preventing a neurological disease, wherein the nucleic acid comprises at least 65% sequence identity to SEQ ID NO: 1. Also disclosed herein are methods of treating, inhibiting, reducing, decreasing, ameliorating, and/or preventing a neurological disease, wherein the nucleic acid comprises at least 70% sequence identity to SEQ ID NO: 1. Also disclosed herein are methods of treating, inhibiting, reducing, decreasing, ameliorating, and/or preventing a neurological disease, wherein the nucleic acid comprises at least 75% sequence identity to SEQ ID NO: 1. Also disclosed herein are methods of treating, inhibiting, reducing, decreasing, ameliorating, and/or preventing a neurological disease, wherein the nucleic acid comprises at least 80% sequence identity to SEQ ID NO: 1. Also disclosed herein are methods of treating, inhibiting, reducing, decreasing, ameliorating, and/or preventing a neurological disease, wherein the nucleic acid comprises at least 85% sequence identity to SEQ ID NO: 1. Also disclosed herein are methods of treating, inhibiting, reducing, decreasing, ameliorating, and/or preventing a neurological disease, wherein the nucleic acid comprises at least 90% sequence identity to SEQ ID NO: 1. Also disclosed herein are methods of treating, inhibiting, reducing, decreasing, ameliorating, and/or preventing a neurological disease, wherein the nucleic acid comprises at least 95% sequence identity to SEQ ID NO: 1. Also disclosed herein are methods of treating, inhibiting, reducing, decreasing, ameliorating, and/or preventing a neurological disease, wherein the nucleic acid comprises at least 99% sequence identity to SEQ ID NO: 1. Also disclosed herein are methods of treating, inhibiting, reducing, decreasing, ameliorating, and/or preventing a neurological disease, wherein the nucleic acid comprises SEQ ID NO: 1.

Also disclosed herein are methods of treating, inhibiting, reducing, decreasing, ameliorating, and/or preventing a neurological disease, wherein the mgll shRNA inhibits expression of an monoacylglycerol lipase (MAGL) protein. In some aspects, the vector further comprises a reporter gene (such as, for example a green fluorescent protein (GFP) gene).

In another aspect, disclosed herein are methods of treating, inhibiting, reducing, decreasing, ameliorating, and/or preventing a neurological disease, wherein the vector further comprises a reporter gene. In some embodiments, the reporter gene is a green fluorescent protein (GFP) gene. In some embodiments, the reporter gene is a luciferase, yellow fluorescent protein (YFP), blue fluorescent protein (BFP), cyane fluorescent protein (CFP), monomeric red fluorescent protein (mRFP), Discosona siriaia (DsRed), mCherry, mOrange, tdTomato, mSTrawberry, mPlum, photoactivatable GFP (PA-GFP), Venus, Kaede, monomeric kusabira orange (mKO), Dronpa, enhanced CFP (ECFP), Emerald, Cyan fluorescent protein for energy transfer (CyPet), super CFP (SCFP), Cerulean, photoswitchable CFP (PS-CFP2), photoactivatable RFP1 (PA-RFP1), photoactivatable mCherry (PA-mCheriy), monomeric teal fluorescent protein (mTFP1), Eos fluorescent protein (EosFP), Dendra, TagBFP, TagRFP, enhanced YFP (EYFP), Topaz, Citrine, yellow fluorescent protein for energy transfer (YPet), super YFP (SYFP), enhanced GFP (EGFP), Superfolder GFP, T-Sapphire, Fucci, mKO2, mOrange2, mApple, Sirius, Azurite, EBFP, and/or EBFP2.

In another aspect, disclosed herein is a method of treating, inhibiting, reducing, decreasing, ameliorating, and/or preventing a neurological disease, wherein the vector crosses a blood-brain-barrier (BBB). In another aspect, disclosed herein is a method of treating, inhibiting, reducing, decreasing, ameliorating, and/or preventing a neurological disease, wherein the vector targets an astrocyte. In another aspect, disclosed herein is a method of treating, inhibiting, reducing, decreasing, ameliorating, and/or preventing a neurological disease, wherein the vector does not target a neuron.

In another aspect, disclosed herein are methods of treating, inhibiting, reducing, decreasing, ameliorating, and/or preventing a neurological disease, wherein a pharmaceutically effective amount of the vector is administered through a bodily fluid of the subject. In some embodiments, the bodily fluid comprises cerebrospinal fluid (CSF). In some embodiments, the bodily fluid comprises blood.

In another aspect, disclosed herein are methods of treating, inhibiting, reducing, decreasing, ameliorating, and/or preventing a neurological disease, wherein the vector is further administered by an intravenous injection, an intracerebroventricular (ICV) injection, an intrathecal injection, or a nasal application. In some embodiments, the vector is further administered by a subcutaneous injection, an intramuscular injection, or any other injection routes.

In another aspect, disclosed herein are methods of treating, inhibiting, reducing, decreasing, ameliorating, and/or preventing a neurological disease, wherein the subject is a mammal. In some embodiments, the mammal is a human or a non-human primate.

In one aspect, disclosed herein is a methods of improving, increasing, retaining, and/or recovering spatial learning and memory retention in a subject with a neurological disease (such as for example, a neurodegenerative disease including, but not limited to Alzheimer's disease, Parkinson's disease, Huntington's disease, Amyotrophic lateral sclerosis (ALS), Friedreich ataxia, Lewy body disease, Spinal muscular atrophy, Multiple sclerosis (MS), Batten disease, Creutzfeldt-Jakob disease, prion diseases, and/or neuropsychiatric disorders, including seizure/epilepsy and mood disorders) or traumatic brain injury in a subject in need thereof, wherein the method comprises administering to the subject any of the engineered AAV vectors disclosed herein. For example, disclosed herein are methods of improving, increasing, retaining, and/or recovering spatial learning and memory retention in a subject with a neurological disease (such as for example, a neurodegenerative disease including, but not limited to Alzheimer's disease, Parkinson's disease, Huntington's disease, Amyotrophic lateral sclerosis (ALS), Friedreich ataxia, Lewy body disease, Spinal muscular atrophy, Multiple sclerosis (MS), Batten disease, Creutzfeldt-Jakob disease, prion diseases, and/or neuropsychiatric disorders, including seizure/epilepsy and mood disorders) or traumatic brain injury in a subject in need thereof, wherein the method comprises administering to the subject an engineered adeno-associated virus (AAV) vector (such as, for example an AAV9 vector) comprising a brain specific promoter (such as for example an astrocyte-specific promoter, including, but not limited to a glial fibrillary acidic protein (gfap) promoter) and a nucleic acid encoding a monoacylglycerol lipase (mgll) double-stranded interfering RNA (such as, for example, a microRNA (miRNA), a small/short interfering RNA (siRNA), a short hairpin RNA (shRNA), or any variation of interfering RNA molecules, including, but not limited to the double-stranded interfering RNA is a mgll miRNA, a mgll siRNA, or a mgll shRNA).

Another example, disclosed herein are methods of improving, increasing, retaining, and/or recovering spatial learning and memory retention in a subject with a neurological disease (such as for example, a neurodegenerative disease including, but not limited to Alzheimer's disease, Parkinson's disease, Huntington's disease, Amyotrophic lateral sclerosis (ALS), Friedreich ataxia, Lewy body disease, Spinal muscular atrophy, Multiple sclerosis (MS), Batten disease, Creutzfeldt-Jakob disease, prion diseases, and/or neuropsychiatric disorders, including seizure/epilepsy and mood disorders) or traumatic brain injury in a subject in need thereof, wherein the method comprises administering to the subject an engineered lentiviral (LV) vector (including, but not limited to Human Immunodeficiency Virus-I (HIV-1), HIV-2, Simian immunodeficiency virus (SIV), feline immunodeficiency virus (FIV)) comprising a brain specific promoter (such as for example an astrocyte-specific promoter, including, but not limited to a glial fibrillary acidic protein (gfap) promoter) and a nucleic acid encoding a monoacylglycerol lipase (mgll) double-stranded interfering RNA (such as, for example, a microRNA (miRNA), a small/short interfering RNA (siRNA), a short hairpin RNA (shRNA), or any variation of interfering RNA molecules, including, but not limited to the double-stranded interfering RNA is a mgll miRNA, a mgll siRNA, or a mgll shRNA).

Disclosed herein are methods of improving, increasing, retaining, and/or recovering spatial learning and memory retention in a subject of any preceding aspect, wherein the nucleic acid comprises at least 50% sequence identity to SEQ ID NO: 1. Also disclosed herein are methods of improving, increasing, retaining, and/or recovering spatial learning and memory retention in a subject of any preceding aspect, wherein the nucleic acid comprises at least 55% sequence identity to SEQ ID NO: 1. Also disclosed herein are methods of improving, increasing, retaining, and/or recovering spatial learning and memory retention in a subject of any preceding aspect, wherein the nucleic acid comprises at least 60% sequence identity to SEQ ID NO: 1. Also disclosed herein are methods of improving, increasing, retaining, and/or recovering spatial learning and memory retention in a subject of any preceding aspect, wherein the nucleic acid comprises at least 65% sequence identity to SEQ ID NO: 1. Also disclosed herein are methods of improving, increasing, retaining, and/or recovering spatial learning and memory retention in a subject of any preceding aspect, wherein the nucleic acid comprises at least 70% sequence identity to SEQ ID NO: 1. Also disclosed herein are methods of improving, increasing, retaining, and/or recovering spatial learning and memory retention in a subject of any preceding aspect, wherein the nucleic acid comprises at least 75% sequence identity to SEQ ID NO: 1. Also disclosed herein are methods of improving, increasing, retaining, and/or recovering spatial learning and memory retention in a subject of any preceding aspect, wherein the nucleic acid comprises at least 80% sequence identity to SEQ ID NO: 1. Also disclosed herein are methods of improving, increasing, retaining, and/or recovering spatial learning and memory retention in a subject of any preceding aspect, wherein the nucleic acid comprises at least 85% sequence identity to SEQ ID NO: 1. Also disclosed herein are methods of improving, increasing, retaining, and/or recovering spatial learning and memory retention in a subject of any preceding aspect, wherein the nucleic acid comprises at least 90% sequence identity to SEQ ID NO: 1. Also disclosed herein are methods of improving, increasing, retaining, and/or recovering spatial learning and memory retention in a subject of any preceding aspect, wherein the nucleic acid comprises at least 95% sequence identity to SEQ ID NO: 1. Also disclosed herein are methods of improving, increasing, retaining, and/or recovering spatial learning and memory retention in a subject of any preceding aspect, wherein the nucleic acid comprises at least 99% sequence identity to SEQ ID NO: 1. Also disclosed herein are methods of improving, increasing, retaining, and/or recovering spatial learning and memory retention in a subject of any preceding aspect, wherein the nucleic acid comprises SEQ ID NO: 1.

Also disclosed herein are improving, increasing, retaining, and/or recovering spatial learning and memory retention in a subject with a neurological disease, wherein the mgll shRNA inhibits expression of an monoacylglycerol lipase (MAGL) protein.

In some aspects, disclosed herein are methods of improving, increasing, retaining, and/or recovering spatial learning and memory retention the vector further comprises a reporter gene (such as, for example a green fluorescent protein (GFP) gene), wherein the vector further comprises a reporter gene. In some embodiments, the reporter gene is a green fluorescent protein (GFP) gene. In some embodiments, the reporter gene is a luciferase, yellow fluorescent protein (YFP), blue fluorescent protein (BFP), cyane fluorescent protein (CFP), monomeric red fluorescent protein (mRFP), Discosoina striata (DsRed), mCherry, mOrange, tdTomato, mStrawberry, mPlum, photoactivatable GFP (PA-GFP), Venus, Kaede, monomeric kusabira orange (mKO), Dronpa, enhanced CFP (ECFP), Emerald, Cyan fluorescent protein for energy transfer (CyPet), super CFP (SCFP), Cerulean, photoswitchable CFP (PS-CFP2), photoactivatable RFP1 (PA-RFP1), photoactivatable mCherry (PA-mCherry), monomeric teal fluorescent protein (mTFP1), Eos fluorescent protein (EosFP), Dendra, TagBFP, TagRFP, enhanced YFP (EYFP), Topaz, Citrine, yellow fluorescent protein for energy transfer (YPet), super YFP (SYFP), enhanced GFP (EGFP), Superfolder GFP, T-Sapphire, Fucci, mKO2, mOrange2, mApple, Sirius, Azurite, EBFP, and/or EBFP2.

In another aspect, disclosed herein is a method of improving, increasing, retaining, and/or recovering spatial learning and memory retention in a subject with a neurological disease of any preceding aspect, wherein the vector crosses a blood-brain-barrier (BBB). In another aspect, disclosed herein is a method of improving, increasing, retaining, and/or recovering spatial learning and memory retention in a subject with a neurological disease of any preceding aspect, wherein the vector targets an astrocyte. In another aspect, disclosed herein is a method of improving, increasing, retaining, and/or recovering spatial learning and memory retention in a subject with a neurological disease of any preceding aspect, wherein the vector does not target a neuron.

Also disclosed herein are methods of improving, increasing, retaining, and/or recovering spatial learning and memory retention in a subject with a neurological disease of, wherein a pharmaceutically effective amount of the vector is administered through a bodily fluid of the subject. In another aspect, disclosed herein is a method of any preceding aspect, wherein the vector is further administered by an intravenous injection, an intracerebroventricular (ICV) injection, an intrathecal injection, or a nasal application. In some embodiments, the vector is further administered by a subcutaneous injection, an intramuscular injection, or any other injection routes.

In another embodiment, disclosed herein is a method of any preceding aspect, wherein the method comprises directing a lentiviral vector to deliver a short hairpin RNA with a brain-specific promoter. In some embodiments, the lentiviral vector (LV) is a LV-gfap-mgll-shRNA vector.

### EXAMPLES

To further illustrate the principles of the present disclosure, the following examples are put forth so as to provide those of ordinary skill in the art with a complete disclosure and description of how the compositions, articles, and methods claimed herein are made and evaluated. They are intended to be purely exemplary of the invention and are not intended to limit the scope of what the inventors regard as their disclosure. Efforts have been made to ensure accuracy wuLn respect to numbers (e.g., amounts, temperatures, etc.); however, some errors and deviations should be accounted for. Unless indicated otherwise, temperature is ° C. or is at ambient temperature, and pressure is at or near atmospheric. There are numerous variations and combinations of process conditions that can be used to optimize product quality and performance. Only reasonable and routine experimentation will be required to optimize such process conditions.

### Example 1: Inactivation of MAGL in Astrocytes does not Induce CB1R Desensitization

Earlier experiments revealed that global MAGL inactivation induces functional tolerance of CB1R that causes antagonism of the endocannabinoid system. This effect greatly reduces the therapeutic efficacy of MAGL inhibitors. To determine whether MAGL inhibition-induced CB1R tolerance is cell type specific, the mgll^{flox/flox} mice were crossed with cell type-specific cre mice (sox2-, syn1-, and gfap-cre) to generate mgll-no-cre (WT), total (tKO), neuron-specific (nKO), and astrocyte-specific (aKO) MAGL knockout (KO) mice. Microglia-specific MAGL KO (mKO) mice were not generated, as MAGL does not play a significant role in degrading 2-AG in microglial cells.

Whole-cell patch clamp recording was used to examine CB1R-mediated GABAergic synaptic responses to WIN55,212-2 (WIN), a synthetic cannabinoid, in the hippocampal slices from WT, tKO, nKO, and aKO mice. As shown in FIG. 2, application of WIN induced about 40% of suppression of IPSCs in WT mice, but the suppression was significantly reduced in tKO and nKO mice, indicating CB IR desensitization in total and neuron MAGL KO mice. In contrast, the magnitude of the WIN-induced suppression of IPSCs in aKO mice was similar to that in WT mice. To ensure that WIN-induced suppression of IPSCs in WT mice is mediated via CB1R, Rimonabant (RIM), a CB1R antagonist was used. The WIN-induced suppression of IPSCs is blocked by RIM. These results show that MAGL inactivation-induced functional tolerance of CB1R results from inhibition of 2-AG metabolism in neurons, rather than in astrocytes and that selective inactivation of MAGL in astrocytes does not induce CB1R receptor desensitization.

### Example 2: Inactivation of MAGL in Neurons Impairs Learning and Memory

MAGL has been considered as a therapeutic target for AD based on results showing that inactivation of MAGL reduces AD neuropathology in APP-TG mice. However, it was recently shown that global inactivation of MAGL may induce some adverse effects. Learning and memory in nKO mice are impaired in the Morris water maze (MWM) test. To further confirm whether inactivation of MAGL in neurons impairs cognitive function, the novel object recognition (NOR) test was used to assess memory retention in WT, tKO, nKO, and aKO mice. As shown in FIG. 3A, memory retention in nKO mice is impaired in the NOR test. To exclude the possibility that this deficit in nKO mice is due to developmental effects of MAGL deletion, AAV9-cre vectors with a synapsin promoter were injected in mgll^{flox/flox} mice at 3 months of age to inactivate MAGL in neurons (FIG. 3B). Cognitive functions in these mice were assessed using both NOR and MWM tests. As shown in FIGS. 3C and 3D, learning and memory in these mice were impaired 30 days after injection of AAV9-Syn-cre vectors. These results provided evidence that proper 2-AG degradation in neurons is required for maintaining normal cognitive function. This raises a concern of whether global inactivation MAGL by pharmacological inhibitors is an ideal approach for an AD therapy, as that will disrupt 2-AG degradation in neurons as well as in peripheral tissues/cells.

### Example 3: Expression of MAGL in Astrocytes is Upregulated in Patients with AD and in AD Animals

It has been shown that activity or expression of MAGL is increased in patients with AD, in APP TG mice or in aging animals. However, no information is available as to whether expression of MAGL in astrocytes is altered. To this end, MAGL expression was detected in astrocytes from postmortem hippocampal tissues of patients with AD, from 6-mo-old 5×FAD APP-TG mice, and from 7-mo-old PS19 tau-TG mice. As shown in FIG. 4, immunoreactivity of MAGL co-localized with GFAP is significantly elevated in the hippocampus of both patients with AD and animal models of AD, showing that 2-AG degradation in astrocytes is likely escalated in AD. Expedited 2-AG breakdown in astrocytes leads to reduction of 2-AG, which is anti-inflammatory and neuroprotective, while elevating 2-AG metabolites prostaglandins and leukotrienes, which are proinflammatory and neurotoxic in astrocytes and surrounding cells. These changes, as a result, promote AD neuropathology.

### Example 4: Overexpression of MAGL in Astrocytes Exacerbates AD Neuropathology and Accelerates Cognitive Deterioration in APP-TG Mice

Since expression of MAGL in astrocytes is elevated in AD, this prompted the determination of whether elevated expression of MAGL in astrocytes promotes AD neuropathology and cognitive decline in APP-TG mice. To do that, human mgll was overexpressed in astrocytes using AAV5 vectors with an astrocytic specific promoter gfap in 3-month-old 5×FAD APP-TG mice (FIGS. 5A and 5B). Overexpression of human MAGL in astrocytes resulted in a robust increase of IL-1β and Aβ42 formation in APP-TG mice (FIG. 5C). As shown in FIGS. 5E and 5F, overexpression MAGL in astrocytes significantly impaired spatial learning and memory retention not only in APP TG mice, but also in WT mice. 5×FAD mice do not display significant impairments in learning and memory at 4 months of age, but approximately around 6 months of ages. This data shows that an increase in expression of MAGL in astrocytes promotes neuroinflammation, exacerbates AD neuropathology, and accelerates cognitive deterioration in APP TG mice.

### Example 5: Inactivation of MAGL in Astrocytes Attenuates Reactivity of Microglia in APP-TG Mice

This example shows that elevated expression of MAGL, which escalates 2-AG breakdown in astrocytes, promotes AD neuropathology and cognitive decline in APP TG mice. The results from a recent TBI study shows that inactivation of MAGL in astrocytes, but not in neurons, produces protective effects against TBI-induced neuropathology. Thus, selective inactivation of MAGL in astrocytes mitigates AD neuropathology and improves synaptic and cognitive function in AD animals. To test this, 5×FAD mice lacking MAGL globally, or specifically in neurons or astrocytes were generated by crossing 5×FAD mice with mgll^{flox/flox}-no-cre (WT), tKO, nKO, and aKO mice, resulting in WT, APP-TG, APP-TG-tKO, APP-TG-nKO, and APP-TG-aKO. Neuroinflammation is one of the neuropathological features in AD. Microglial cells are the most important resident immunocompetent cells that release many proinflammatory factors in the brain. Reactivity of astroglia cells is an important marker for neuroinflammation. To this end, reactivity of microglia in APP-TG mice lacking MAGL was assessed. As shown in FIG. 6, signal intensity for Ibal immunoreactivity is similar in APP-TG and APP-TG-nKO mice, indicating that inactivation of MAGL in neurons does not reduce microglial neuroinflammation in APP-TG mice. However, the Ibal immunoreactivity is significantly reduced in APP-TG-aKO mice, showing that elevated 2-AG in astrocytes as a paracrine signaling molecule acts on microglial cells, leading to suppression of microglia-mediated neuroinflammation. In addition, 2-AG metabolites produced in astrocytes are the primary source of inflammation-associated eicosanoids. Therefore, inactivation of MAGL in astrocytes produces synergistic effects by elevating 2-AG signaling and reducing its metabolites (“a dual hit’ process) in mitigation of inflammatory responses.

### Example 6: Inactivation of MAGL in Astrocytes Reduces Aβ Pathology in APP-TG Mice

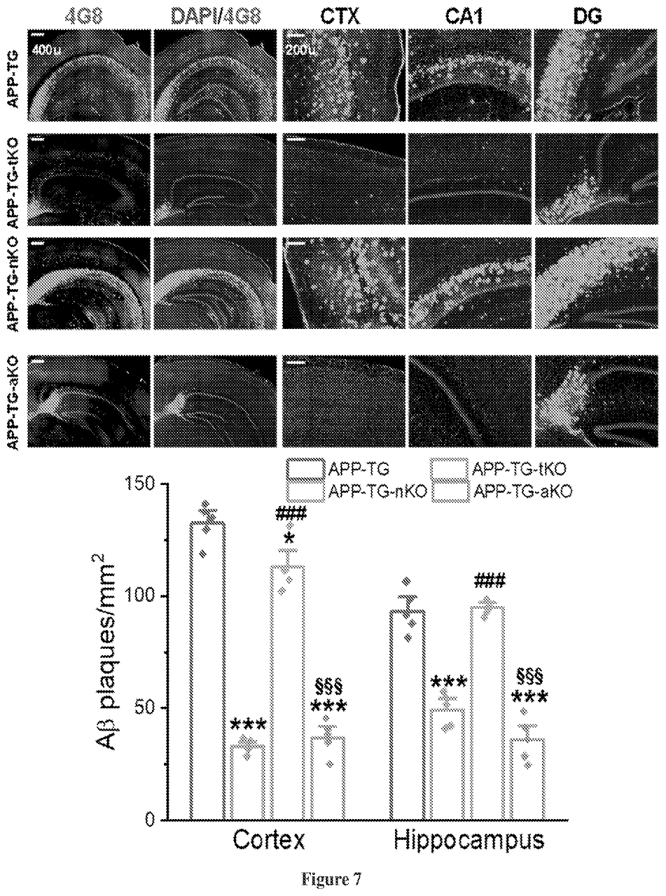

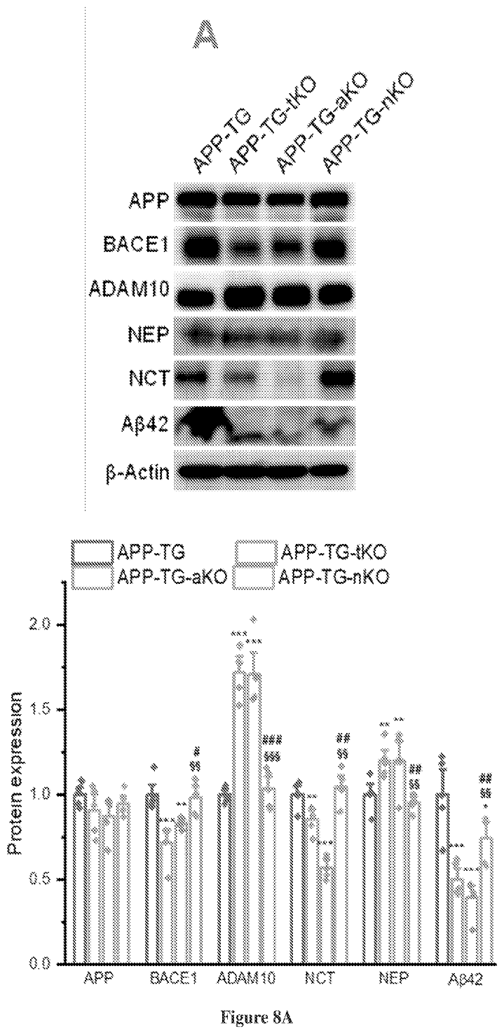

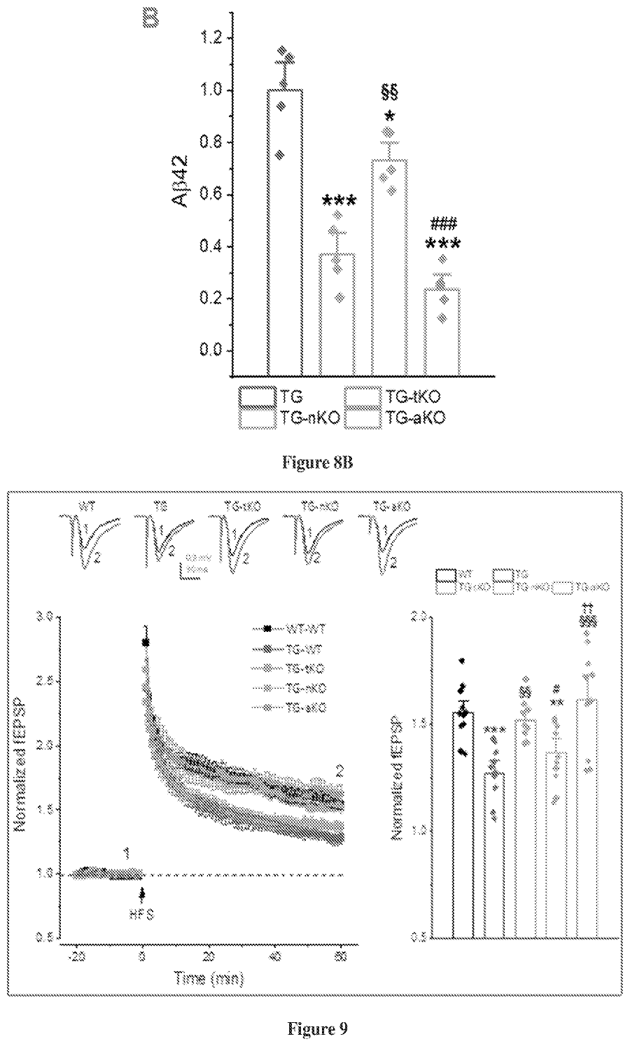

Accumulation and deposition of Aβ are one of the two neuropathological hallmarks of AD. Pharmacological or genetic inactivation of MAGL reduces Aβ in APP-TG mice. To determine whether MAGL inactivation-induced reduction of Aβ accumulation is cell type-specific in APP-TG mice, total Aβ in the brain of 6-month-old APP-TG, APP-TG-tKO, APP-TG-nKO, and APP-TG-aKO mice was assessed. As shown in FIG. 7, APP-TG-tKO and APP-TG-ago mice displayed a decrease in Aβ plaques. However, inactivation of MAGL in neurons only slightly reduced Aβ plaques in the cortex, but not in the hippocampus, showing that reduction of Aβ by global inactivation of MAGL results largely from restraining 2-AG degradation in astrocytes, rather than in neurons. The reduced Aβ plaques in APP-TG-tKO and APP-TG-aKO are associated with decreased Aβ processing. To test this, expression of APP, ADAM10 (α-secretase), BACE1 (β-secretase), nicarstrin (NCT, a component of 7-secretase), neprilysin (NEP), and Aβ42 was assessed using immunoblot in APP-TG mice deficient in MAGL. As shown in FIG. 8A, while expression of ADAM10 was robustly elevated, expression of BACE1 and NCT was significantly reduced in APP-TG-tKO and APP-TG-aKO mice, but not in APP-TG-nKO mice. In addition, ELISA was used to measure Aβ42 and found that Aβ42 is robustly reduced in APP-TG-tKO and APP-TG-aKO mice, but only slightly reduced in APP-TG-nKO mice (FIG. 8B). These data provide important information showing that limiting 2-AG breakdown in astrocytes, but not in neurons, reduces the process of proteolytic cleavage of APP to form Aβ, resulting in reduction of Aβ as shown in FIG. 7.

### Example 7: Inhibition of 2-AG Metabolism in Astrocytes Prevents Impairment of LTP in APP-TG Mice

Inhibition of 2-AG degradation by inactivation of MAGL in astrocytes decreases Aβ pathology in APP-TG mice, showing that inactivation of astrocytic MAGL improves synaptic and cognitive functions in AD. It is generally accepted that memory and cognitive deficits in AD at early stages primarily result from synaptic failure. Therefore, long-term potentiation (LTP) was assessed in WT, APP-TG, APP-TG-tKO, APP-TG-nKO, and APP-TG-aKO mice and it was found that LTP in APP-TG mice was impaired. However, TG-tKO and TG-aKO mice displayed normal LTP when compared with WT mice. In contrast, LTP was impaired in APP-TG-nKO mice (FIG. 9), similar to that in APP-TG mice. These results show that inactivation of MAGL in astrocytes, but not in neurons, prevents deterioration in hippocampal synaptic plasticity in APP TG mice.

### Example 8: Knockdown of Astrocytic MAGL Reduces Tauopathy in PS19 Tau-TG Mice

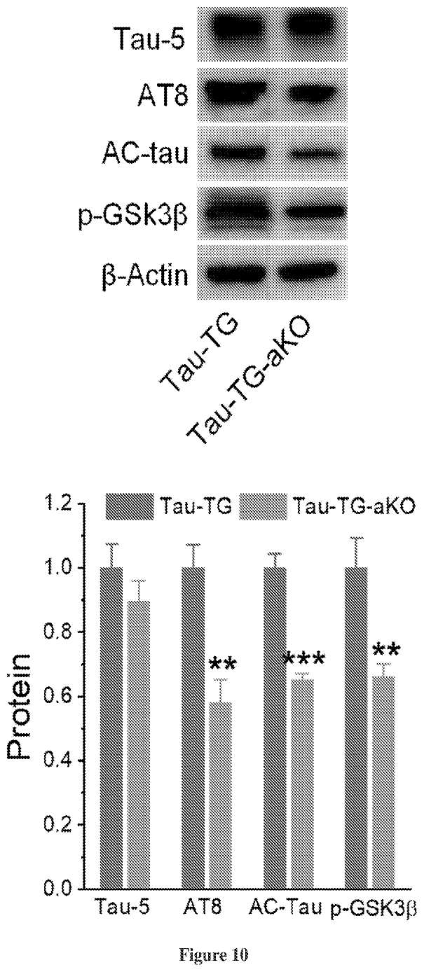

Hyperphosphorylated tau is another hallmark of AD neuropathology. It is crucial to know whether inactivation of MAGL in astrocytes reduces tauopathy. To test this, tau-TG mice lacking MAGL in astrocytes was generated by crossing PS19 tau-TG mice with aKO mice. Total tau (Tau-5), acetylated tau (AC-tau), phosphorylated tau (AT8), and p-GSK3(3 that phosphorylates tau, was assessed in the hippocampus of tau-TG, and tau-TG-aKO mice. As shown in FIG. 10, expression of AC-tau, AT8, and p-GSK3P was significantly reduced in tau-TG-aKO mice. A significant reduction of Tau-5 was not detected as this protein is transgenically expressed. These results show that tau pathology is ameliorated by restraining 2-AG degradation in astrocytes.

### Example 9: Silencing of Astrocytic MAGL by Intrahippocampal Injection of AAV Vectors Prevent Deterioration in Expression of Synaptic Proteins in APP-TG Mice

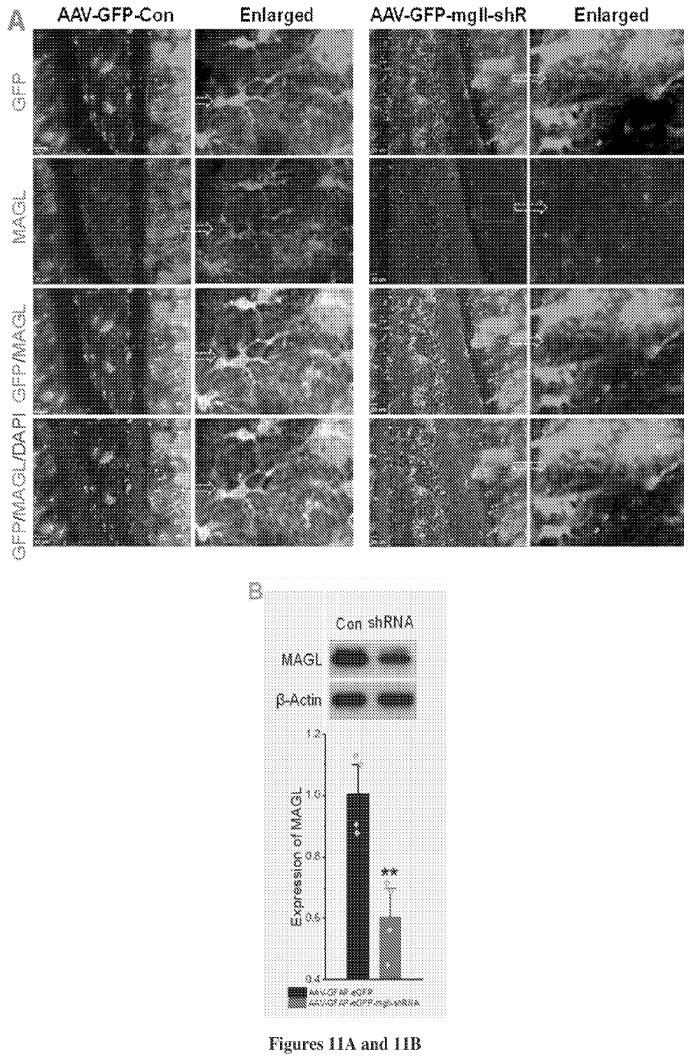

2-AG metabolism in astrocytes promotes neuropathology and drives synaptic and cognitive deterioration in TBI and AD animals (FIGS. 6, 7, 8, 9, and 10). This further shows that the observed neuroprotective effects of global MAGL inactivation in AD animals result largely from inhibition of 2-AG degradation in astrocytes, rather than in neurons. Thus, astrocytic MAGL is an ideal therapeutic target for AD. Nevertheless, current pharmacotherapies lack the capacity to target a gene in a specific type of cells (i.e., astrocytes) in the brain, but global inactivation of MAGL (e.g., pharmacological inhibitors) will also induce some adverse effects, including impairments in cognitive function (FIG. 3). Growing evidence indicates that AAV vector-mediated gene replacement, silencing, and editing technologies provide a platform for the treatment of a variety of human diseases, including neurodegenerative diseases. Recent clinical studies showed that AAV gene therapy is safe and effective. Therefore, the AAV-mediated gene silencing strategy allows targeting of MAGL specifically in astrocytes. To test this, AAV5-GFAP-eGFP-mgll-shRNAmir(2), an AAV vector with an astrocytic promoter gfap, was generated to render MAGL loss-of-function in astrocytes by intrahippocampal injection. As shown in FIG. 11, expression of MAGL in astrocytes was significantly reduced by stereotaxic injection of AAV5-GFAP-eGFP-mgll-shRNA into the hippocampus, showing that the AAV vectors are functional in successfully knocking MAGL down in astrocytes of the hippocampus.

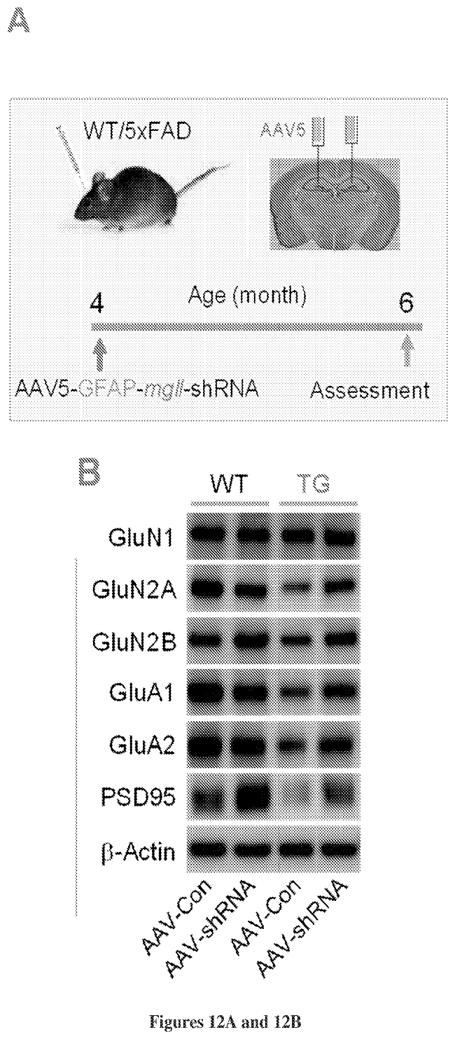

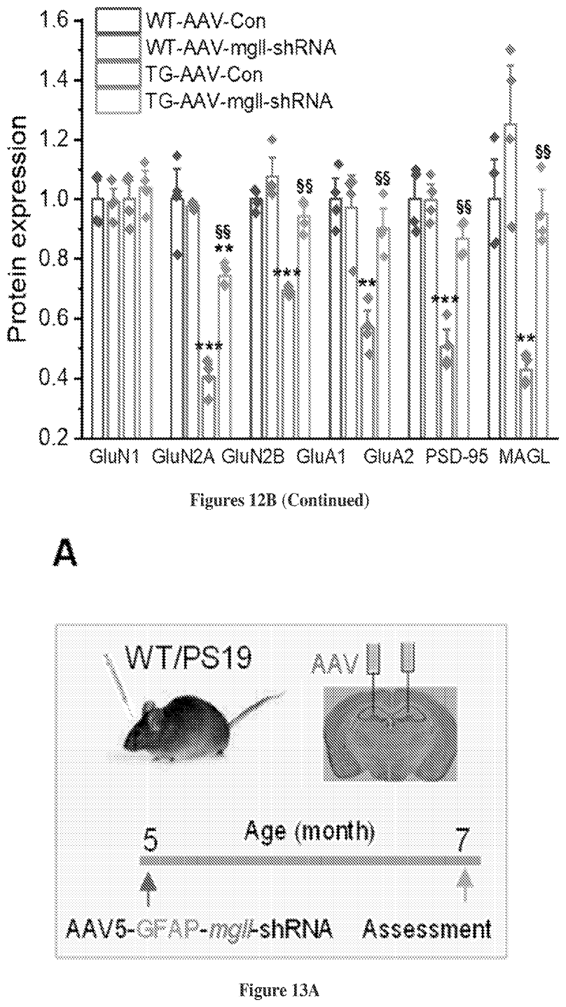

Then, expression of important synaptic proteins in the hippocampus of 6-month-old APP-TG mice that were injected with AAV5-GFAP-mgll-shRNA was assessed. As shown in FIG. 12, knockdown of MAGL in astrocytes prevented deterioration in expression of important synaptic proteins, including PSD95 and glutamate receptor subunits (GluA1, GluA2, GluN2A, and GluN2B) in the hippocampus of APP-TG mice. Expression of these synaptic proteins is significantly downregulated in 6-month-old 5×FAD mice, showing that AAV-mediated silencing of MAGL in astrocytes is capable of maintaining the synaptic integrity in the hippocampus of APP-TG mice.

### Example 10: AAV-Mediated Silencing of Astrocytic MAGL Improves Spatial Learning and Memory Retention in Tau-TG Mice

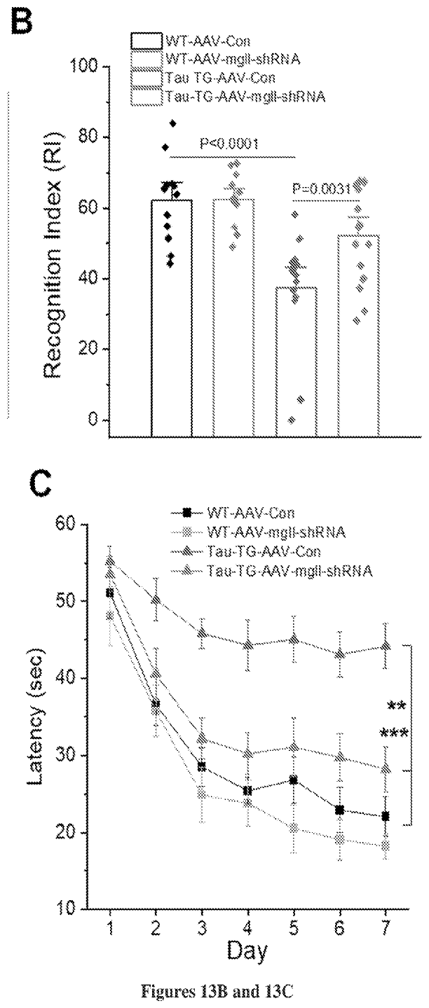

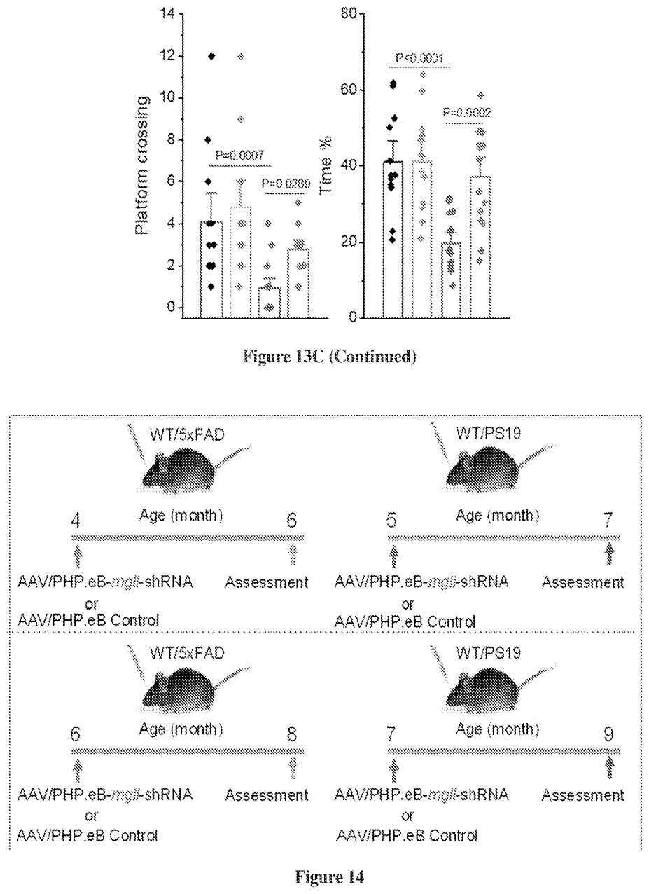

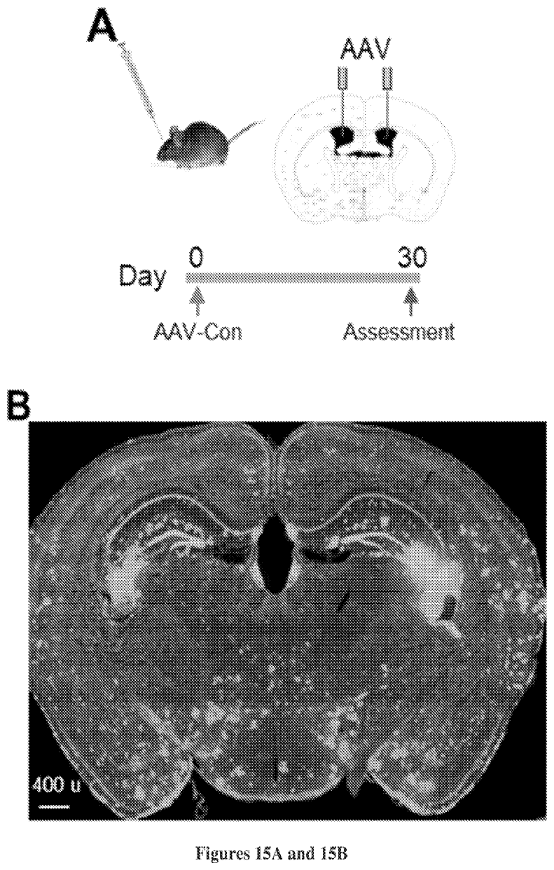

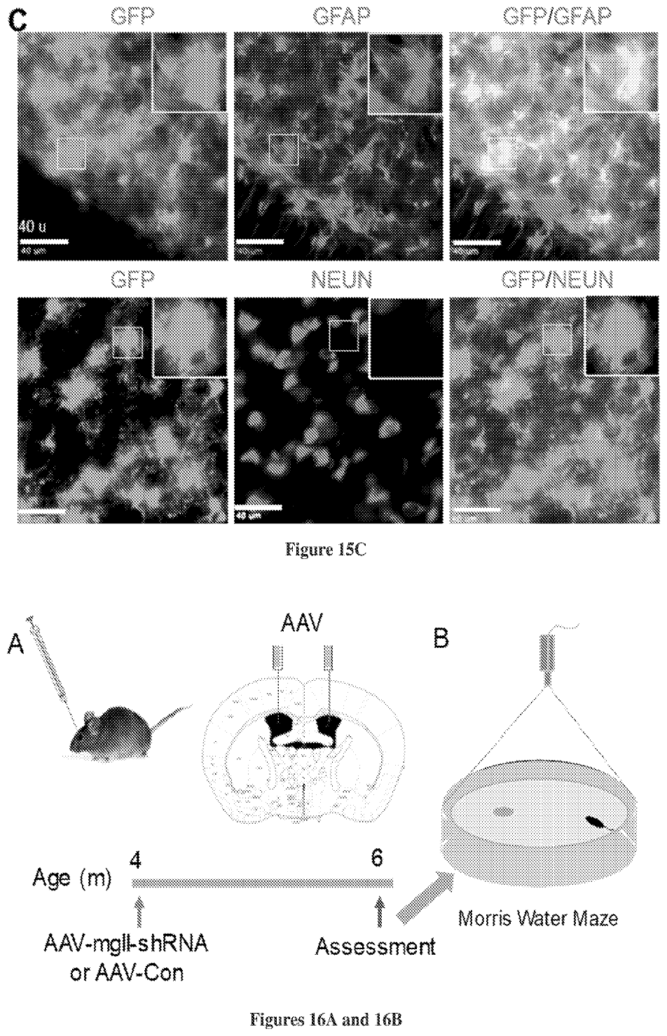

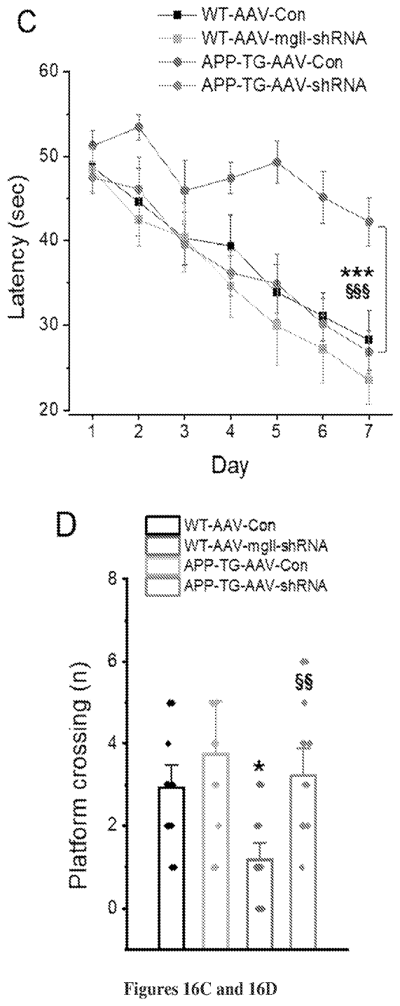

Accumulation and deposition of hyperphosphorylated tau is a neuropathological hallmark of AD. To increase generalizability of AAV-mediated silencing of MAGL in astrocytes as an ideal therapy for AD, PS19 tau-TG mice, a widely used tau mouse model of AD, were used. PS19 tau-TG mice display cognitive deficits starting around 7 months of age. Thus, AAV vectors expressing GFAP-mgll-shRNA or control vectors were stereotaxically injected into the hippocampus of 5-month-old PS19 tau-TG mice. NOR and MWM tests were conducted 2 months after injection of AAV vectors (i.e., at 7 months of age). As shown in FIG. 13. WT animals did not show any significant differences in learning and memory between the mice treated with AAV-GFAP-mgll-shRNA vectors and controls. Learning and memory were significantly impaired in 7-month-old tau-TG mice that received AAV control vectors in both NOR and MWM tests. However, the impairment was prevented in tau-TG mice that received AAV-GFAP-mgll-shRNA vectors, showing that knockdown of MAGL in astrocytes prevents cognitive decline in tau-TG mice. These data provide important information showing that AAV-mediated silencing of MAGL in astrocytes improves synaptic and cognitive functions in both APP- and tau-TG animal models of AD.

Taken together, these results show that inactivation of MAGL in astrocytes, but not in neurons, attenuates neuropathology and improves synaptic and cognitive functions in mouse models of AD, showing that the neuroprotective effects of global MAGL inactivation with pharmacological inhibitors largely results from limiting 2-AG metabolism in astrocytes, rather than in neurons. In particular, global inactivation of MAGL induces some adverse effects resulting from disruption of 2-AG degradation in neurons and in peripheral cells. AAV-mediated gene silencing strategy provides an ideal platform allowing specific MAGL knockdown in astrocytes as a novel therapy for AD. AAV-mediated knockdown of astrocytic MAGL by intrahippocampal injection of vectors in improving synaptic and cognitive functions in AD animals (FIGS. 12 and 13).

### Example 11: Methods

Currently, no single animal model fully represents human AD neuropathology and deficits in cognitive function. 5×FAD APP-TG mice and tau P301S mice have been widely used as Aβ and tau pathology models for AD research in many laboratories. To determine AAV-mediated silencing of MAGL in astrocytes as a novel and efficacious gene therapy for AD, both 5×FAD APP-TG and PS19 tau-TG mice are used as animal models of AD.

MAGL in astrocytes is an ideal therapeutic target for AD. Since pharmacological MAGL inhibitors inactivate MAGL globally, which cause some adverse effects, including cognitive impairments (FIG. 3), the AAV-mediated gene silencing technology is used to knock MAGL down specifically in astrocytes. AAV-mediated knockdown of astrocytic MAGL by direct intrahippocampal injection of AAV5-GFAP-eGFP-mgllmir(2) vectors improved synaptic and cognitive functions in AD animals (FIGS. 12 and 13). However, intrahippocampal injection of AAV vectors is not a clinically practicable approach. In addition, the data generated from intrahippocampal injection of AAV vectors does not represent the results from the administration of AAV vectors through other routes that are frequently used in clinic (e.g., intrathecal, intracerebroventricular, intravenous, or nasal). Herein, to enhance the transduction efficiency, AAV-PHP.eB, a novel variant of AAV9, are used with the same cloning strategy as used to generate AAV-PHP.eB-GFAP-eGFP-mgll-shRNAmir(2) to selectively silence MAGL in astrocytes. AAV-PHP.eB-GFAP-eGFP vectors are used as the control. AAV-PHP.eB vectors have been shown to be able to cross the blood-brain barrier (BBB) by intravenous administration in rodents. It was recently revealed that AAV-PHP.eB is also capable of crossing BBB in non-human primates. AAV-PHP.eB exhibits much higher efficiency in transducing in neurons and astrocytes than AAV9. Thus, AAV-PHP.eB is one of the most efficient AAV vectors in the field for CNS gene transfer.

5×FAD TG mice display impairments in synaptic and cognitive functions starting approximately at 6 months of age, while PS19 mice display synaptic and cognitive deficits starting around 7 months of age. Therefore, astrocytic MAGL is silenced by a single ICV injection of AAV-PHP.eB-GFAP-eGFP-mgll-shRNAmir(2) or AAV-PHP.eB-GFAP-eGFP control vectors in 5×FAD APP-TG mice at 4 months of age and PS19 tau-TG mice at 5 months of age before they display synaptic and cognitive deficits (FIG. 14). Neuropathology, synaptic and cognitive functions are assessed 2 months after administration of AAV vectors when the animals display impairments in synaptic and cognitive functions. Next, astrocytic MAGL is silenced by a single ICV injection of AAV-PHP.eB-GFAP-eGFP-mgll-shRNAmir(2) in APP-TG mice at 6 months of age and tau-TG mice at 7 months of age after the animals display synaptic and cognitive deficits (FIG. 14). Neuropathology, synaptic and cognitive functions are assessed 2 months after administration of AAV vectors. ICV injections of AAV-PHP.eB vectors expressing mgll-shRNA or the control with a GFAP promoter are achieved using a 5 μl Hamilton syringe with a 32 ga needle under stereotaxic guidance (at these coordinates: anteroposterior, −0.5; mediolateral, ±1.0; and dorsoventral, −2.3).

Blind testing: All experiments are performed in a blinded fashion to prevent any scientific bias in the experiments. For example, animals are randomly assigned to the experimental groups, and the experimenters do not know the identity of the groups, genotypes, or the treatments.

Sex as a biological variable: Data from both sexes are included. If there are differences in changes in neuropathology, synaptic and cognitive functions between males and females, animals are then grouped and analyzed, and the number of animals adjusted accordingly.

### Example 12: AAV-Mediated Silencing of MAGL in Astrocytes Alleviates AD Neuropathology and Prevents Synaptic and Cognitive Deterioration in Both APP- and Tau-TG Mice

AD is the most common causes of dementia in the elderly. Unfortunately, there are no effective therapies available for prevention and treatment of AD. MAGL is a therapeutic target for AD. However, it was recently shown that global inactivation of MAGL induces some adverse effects, including cognitive impairments (FIG. 3), which were not noticed previously. This shows that global inactivation of MAGL (e.g., pharmacological MAGL inhibitors) does not achieve ideal therapeutic goals for AD. Results showed that expression of MAGL in astrocytes is elevated in patients with AD and animal models of AD (FIG. 4) and that selective inactivation of MAGL in astrocytes, but not in neurons, mitigates neuropathology and improve synaptic and cognitive functions in AD animals (FIGS. 6, 7, 8, and 9). These results show that selective inactivation of MAGL in astrocytes achieves better therapeutic effects for AD. To specifically silence MAGL in astrocytes, an AAV vector-mediated gene silencing strategy is used to knock MAGL down in astrocytes. For prevention of developing AD or for early intervention, AAV-PHP.eB-GFAP-eGFP-mgll-shRNAmir(2) vectors are administered into the cerebrospinal fluid (CSF) by a single ICV injection to selectively silence astrocytic MAGL in 4-month-old 5×FAD APP-TG mice or 5-month-old PS19 tau-TG mice before the animals display synaptic and cognitive deficits, as depicted in FIG. 14. Neuropathology, synaptic and cognitive functions are assessed 2 months after administration of AAV vectors.

2-AG and its metabolites in APP- and tau-TG mice injected with AAV-GFAP-mgll-shRNA vectors. 2-AG in neurons and astrocytes is predominantly hydrolyzed by MAGL. It is important to know how much changes in the contents of 2-AG in the brain of APP-TG and tau-TG mice after knocking MAGL down in astrocytes. LC-MS/MS mass spectrometry is used to measure brain region-specific (the cortex, hippocampus, and cerebellum) 2-AG levels and its metabolites, including arachidonic acid (AA), PGE2, and 12- or 15-HETE in APP-TG, tau-TG mice, and their age-matched WT mice that receive AAV-PHP.eB-GFAP-eGFP-mgll-shRNA or control vectors. In addition, immunoblot and/or immunostaining is also used to assess expression of MAGL in astrocytes of different brain regions from the animals treated with the AAV vectors.

Neuropathology in APP- and tau-TG mice that receive AAV-GFAP-mgll-shRNA vectors. Senile plaques, which are extracellular accumulation and deposition of Aβ plaques, and neurofibrillary tangles (NFT), which are intracellular deposition of hyperphosphorylated tau proteins, are the two neuropathological hallmarks of AD. In addition, neuroinflammation is one of the important neuropathological features in the context of AD. Therefore, qPCR, ELISA, IHC, and Western blot (WB) analyses are used to assess neuroinflammation, Aβ and tau pathologies. In APP-TG mice and their age-matched WT mice that receive AAV-PHP.eB-GFAP-mgll-shRNA or control vectors, the following are assessed: a) proinflammatoiy cytokines (IL-1β, IL-6 and TNFα), reactivity of astrocytes and microglia by detecting immunoreactivity of GFAP and Iba-1, and phosphorylated NF-kB (p-NF-kB); b) expression of the enzymes that synthesize Aβ, including BACE1 (β-secretase), Pen2 and nicastrin (γ-secretase), or the enzymes that degrade Aβ or prevent Aβ formation, including ADAM-10 (α-secretase), neprilysin (NEP), and insulin degrading enzyme (IDE); and c) total Aβ and Aβ42 (plaques and soluble AD). In tau-TG mice that receive AAV-PHP.eB-GFAP-mgll-shRNA vectors, the following ware assessed: a) proinflammatory cytokines, reactivity of astrocytes and microglia, and p-NF-kB; b) total tau (Tau-5), acetylated tau (AC-tau), and phosphorylated tau (p-tau), including p-tau Thr181 (p-tauT181) and p-tau Ser202/Thr205 (AT8), and c) Cdk5, p35/25, and p-GSK3P that phosphorylate tau.

Degenerating neurons in APP- and tau-TG mice treated with AAV-GFAP-mgll-shRNA. Neurodegeneration is an important characteristic marker in AD. Herein, silencing of MAGL in astrocytes to protect neurons from degeneration in APP- and tau-TG mice is assessed. Degenerating neurons in the hippocampus is detected by Fluoro-Jade C (FJC) staining (a specific marker for degenerating neurons) in brain sections from WT, APP- and tau-TG mice that receive AAV-PHP-eB-GFAP-eGFP-mgll-shRNA or control vectors.

The integrity of synaptic structure and function in APP- and tau-TG mice that receive AAV-GFAP-mgll-shRNA. Synaptic structure and function are highly plastic, which are crucial for information processing and encoding memory in the brain. Therefore, the integrity of synaptic structure and function in APP- and tau-TG mice and their age-matched WT mice that receive AAV-PHP-eB-GFAP-eGFP-mgll-shRNA or control vectors is determined. The following are determined: a) expression of AMPA and NMDA glutamate receptor subunits, including GluAL GluA2, GluN1, GluN2A, and GluN2B, and pre- and post-synaptic markers synaptophysin (Syn) and PSD-95, in the hippocampus is assessed using immunoblot or immunostaining; b) hippocampal basal synaptic transmission in terms of input-output function by increment of stimulus intensity and LTP induced by high-frequency stimulation (HFS) or by theta-burst stimulation (TBS) at CA3-CA1 synapses or perforant path synapses in the dentate gyrus; and c) morphology of dendritic spines in terms of the density and shapes (e.g., thin, mushroom, or stubby) on apical dendrites of hippocampal neurons using the Golgi staining.

Learning and memory in APP- and tau-TG mice injected with AAV-GFAP-mgll-shRNA. Learning and memory are the most important cognitive function. Loss of memory is a characteristic symptom in AD. Behavioral assessments are conducted, including the classical Morris water maze (MWM) and the novel object recognition (NOR) tests in APP- and tau-TG mice and their WT mice that receive AAV-PHP-eB-GFAP-eGFP-mgll-shRNA or control vectors.

### Example 13: Reversal or Slowing of AD Neuropathology and Synaptic and Cognitive Declines in Ad Animals with AAV-Mediated Astrocyte-Specific Silencing of MAGL

It is assessed whether knockdown of MAGL in astrocytes by administration of AAV vectors into the CSF reduces AD neuropathology and prevents synaptic and cognitive deterioration in APP- and tau-TG mice before the animals display symptoms of synaptic and cognitive deficits. It is also determined whether AAV-mediated silencing of astrocytic MAGL is a potential therapy for treating AD or halting progression of the disease. To this end, MAGL is knockdown in astrocytes in APP- and tau-TG mice at ages (at 6 months of age for APP-TG mice and 7-months of age for tau-TG mice, respectively) when they already display synaptic and cognitive impairments (FIG. 14). AD neuropathology, synaptic and cognitive functions are assessed 2 months after ICV injection of AAV-PHP-eB-GFAP-eGFP-mgll-shRNA vectors.

AD neuropathology in APP- and tau-TG mice treated with AAV vectors expressing GFAP-mgll-shRNA. As mentioned above, qPCR, ELISA, IHC, and Western blot (WB) analyses are used to assess neuroinflammation. Aβ and tau pathologies. In APP-TG mice and their age-matched WT mice receive AAV-PHP.eB-GFAP-mgll-shRNA or control vectors to assess the following: a) cytokines (IL-1β, IL-6 and TNFα), reactivity of astrocytes and microglia, and p-NF-kB; b) expression of the enzymes, including BACE1, Pen2 and nicastrin. ADAM-10, neprilysin (NEP), and (IDE); and c) total Aβ and Aβ42. In tau-TG mice and their age-matched WT mice that receive AAV-PHP.eB-GFAP-mgll-shRNA or control vectors, the following is also assessed: a) cytokines, reactivity of astrocytes and microglia, and p-NF-kB; b) Tau-5, AC-tau, and p-tau, including p-tau Thr181 (p-tauT181) and p-tau Scr202/Thr205 (AT8), and c) Cdk5, p35/25, and p-GSK3P.

Degenerating neurons in APP- and tau-TG mice treated with AAV-GFAP-mgll-shRNA. Astrocyte-specific silencing of MAGL assesses whether the neurodegenerative process is slowed in APP- and tau-TG mice. Degenerating neurons in the hippocampus are detected by FJC staining in brain sections from WT, APP- and tau-TG mice that receive AAV-PHP-eB-GFAP-eGFP-mgll-shRNA or control vectors.

Integrity of synaptic structure and function in APP- and tau-TG mice that receive AAV-GFAP-mgll-shRNA. The following are also assessed: a) expression of glutamate receptor subunits (GluA1, GluA2, GluN1, GluN2A, and GluN2B), Syn, and PSD-95; b) hippocampal basal synaptic transmission and LTP; c) morphology of dendritic spines in APP- and tau-TG mice and their age-matched WT mice that receive AAV-PHP-eB-GFAP-eGFP-mgll-shRNA or control vectors.

Learning and memory in APP- and tau-TG mice injected with AAV-GFAP-mgll-shRNA. MWM and NOR tests in APP- and tau-TG mice and their age-matched WT mice that receive AAV-PHP-eB-GFAP-eGFP-mgll-shRNA or control vectors are performed.

It will be apparent to those skilled in the art that various modifications and variations can be made in the present disclosure without departing from the scope or spirit of the invention. Other embodiments of the disclosure will be apparent to those skilled in the art from consideration of the specification and practice of the methods disclosed herein. It is intended that the specification and examples be considered as exemplary only, with a true scope and spirit of the invention being indicated by the following claims.

1. An engineered adeno-associated virus (AAV) vector comprising an astrocyte-specific promoter and a nucleic acid encoding a monoacylglycerol lipase (mgll) shRNA.

2. The engineered AAV vector of claim 1, wherein the nucleic acid comprises at least 70% sequence identity to SEQ ID NO: 1.

3. (canceled)

4. (canceled)

5. (canceled)

6. The engineered AAV vector of claim 1, wherein the astrocyte-specific promoter is a glial fibrillary acidic protein (gfap) promoter.

7. The engineered AAV vector of claim 1, wherein the mgll shRNA inhibits expression of an monoacylglycerol lipase (MAGL) protein.

8. (canceled)

9. (canceled)

10. (canceled)

11. The engineered AAV vector of claim 1, wherein the vector crosses a blood-brain-barrier (BBB).

12. A method of preventing or treating a neurological disease or traumatic brain injury in a subject in need thereof, wherein the method comprises administering to the subject the engineered AAV vector of claim 1.

13-23. (canceled)

24. The method of claim 12, wherein the vector targets an astrocyte.

25. The method of claim 12, wherein the vector does not target a neuron.

26. (canceled)

27. (canceled)

28. The method of claim 12, wherein the neurological disease is a neurodegenerative disease.

29. The method of claim 28, wherein the neurodegenerative disease comprises Alzheimer's disease, Parkinson's disease, Huntington's disease, or Amyotrophic lateral sclerosis (ALS), Multiple sclerosis (MS), Frontotemporal dementia (FTD), Chronic traumatic encephalopathy (CTE), seizures/epilepsy, and mood disorders.

30. (canceled)

31. (canceled)

32. A method of improving spatial learning or memory retention in a subject with a neurodegenerative disease or traumatic brain injury disease comprising administering to the subject the engineered AAV vector of claim 1.

33-45. (canceled)

46. The method of claim 32, wherein the vector targets an astrocyte.

47. The method of claim 32, wherein the vector does not target a neuron.

48. (canceled)

49. (canceled)

50. The method of claim 53, wherein the neurodegenerative disease comprises Alzheimer's disease, Parkinson's disease, Huntington's disease, or Amyotrophic lateral sclerosis (ALS), Multiple sclerosis (MS), Frontotemporal dementia (FTD), Chronic traumatic encephalopathy (CTE), seizure/epilepsy, and mood disorders.

51-52. (canceled)

53. The method of claim 32, wherein the neurological disease is a neurodegenerative disease.
